SEQUENCE LISTING (23 sequences)

SEQ ID NO: 1 - protein, 9 aa, synthetic construct
CAEECRPIA

SEQ ID NO: 2 - protein, 15 aa, synthetic construct
GEPPPGKPADDAGLV

SEQ ID NO: 3 - protein, 9 aa, synthetic construct
SLLGRMKGA

SEQ ID NO: 4 - protein, 29 aa, synthetic construct
YADAIFTNSYRKVLGQLSARKLLQDIMSR

SEQ ID NO: 5 - protein, 9 aa, synthetic construct
WAGGDASGE

SEQ ID NO: 6 - protein, 4 aa, synthetic construct
RKLH

SEQ ID NO: 7 - protein, 4 aa, synthetic construct
AWFK

SEQ ID NO: 8 - protein, 5 aa, synthetic construct
WAWFK

SEQ ID NO: 9 - protein, 29 aa, synthetic construct
YADAIFTNSYRKVLGQLSARKLLQDIMSR

SEQ ID NO: 10 - protein, 24 aa, synthetic construct
MAPRGFSCLLLLTSEIDLPVKRRA

SEQ ID NO: 11 - protein, 10 aa, synthetic construct
YNWNSFGLRF

SEQ ID NO: 12 - protein, 37 aa, synthetic construct
LLGDFFRKSKEKIGKEFKRIVQRIKDFLRNLVPRTES

SEQ ID NO: 13 - protein, 16 aa, synthetic construct
MRWQEMGYIFYPRKLR

SEQ ID NO: 14 - protein, 4 aa, synthetic construct
AEDG

SEQ ID NO: 15 - protein, 8 aa, synthetic construct
TPLVTLFK

SEQ ID NO: 16 - protein, 7 aa, synthetic construct
VRSSSRT

SEQ ID NO: 17 - protein, 32 aa, synthetic construct
PPLSQETFSDLWKLLKKWKMRRNQFWVKVQRG

SEQ ID NO: 18 - protein, 27 aa, synthetic construct
ETFSDLWKLLKKWKMRRNQFWVKVQRG

SEQ ID NO: 19 - protein, 20 aa, synthetic construct
KRRQTSMTDFYHSKRRLIFS

SEQ ID NO: 20 - protein, 28 aa, synthetic construct
SDAAVDTSSEITTKDLKEKKEVVEEAEN

SEQ ID NO: 21 - protein, 28 aa, synthetic construct
HSDAVFTDNYTRLRKQMAVKKYLNSILN

SEQ ID NO: 22 - protein, 43 aa, synthetic construct
SDKPDMAEIEKFDKSKLKKTETQEKNPLPSKETIEQEKQAGES

SEQ ID NO: 23 - protein, 9 aa, synthetic construct
KTKKEAAKK

(19) United States
(12) Patent Application Publication
(10) Pub. No.: US 2026/0053928 A1
(43) Pub. Date: Feb. 26, 2026

(54) SYNERGISTIC COMBINATION OF NAD+ AND THERAPEUTIC PEPTIDES DELIVERED VIA IONTOPHORESIS FOR ENHANCED AND PROLONGED REGENERATIVE EFFECTS

(71) Applicants: Nicholas Jerome Andrews, Flanders, NJ (US); Roy Charles Korth, Grove City, OH (US); Charles David Sly, Grove City (OH)

(72) Inventors: Nicholas Jerome Andrews, Flanders, NJ (US); Roy Charles Korth, Grove City, OH (US); Charles David Sly, Grove City (OH)

(21) Appl. No.: 19/383,710

(22) Filed: Nov. 9, 2025

Related U.S. Application Data
(63) Continuation-in-part of application No. 19/186,065, filed on Apr. 22, 2025.
(60) Provisional application No. 63/636,901, filed on Apr. 22, 2024.

(51) Int. Cl. A61K 41/00; A61K 31/7084; A61K 38/06; A61K 47/12; A61M 37/00; A61N 1/30

(52) U.S. Cl. CPC A61K 41/0047; A61K 31/7084; A61K 38/06; A61K 47/12; A61M 37/0015; A61N 1/303; A61M 2037/0007; A61M 2037/0023

(57) ABSTRACT

The present disclosure also provides for a composition comprising a combination of (a) NAD+, and (b) one or more additional therapeutic compounds, wherein one or more additional therapeutic compounds are compatible with transdermal delivery, such as iontophoretic delivery, while providing minimal skin irritation.

### CROSS-REFERENCE TO RELATED APPLICATIONS

This application is a continuation-in-part of U.S. patent application Ser. No. 19/186,065 filed on Apr. 22, 2025, which application claims the benefit of the filing date of U.S. Provisional Application No. 63/614,144 filed on Dec. 22, 2023, the disclosures of which are hereby incorporated by reference herein in their entireties.

### SEQUENCE LISTING

The contents of the electronic sequence listing (PushPatch.xml sequence listing.xml; Size: 20,808 bytes; and Date of Creation: Apr. 21, 2025) is herein incorporated by reference in its entirety.

### FIELD OF DISCLOSURE

The present disclosure relates to the field of regenerative medicine and drug delivery systems. Specifically, the present disclosure pertains to the delivery of nicotinamide adenine dinucleotide (NAD+) in combination with one or more therapeutic peptides, peptide bioregulators, or compounds that are believed to facilitate cellular respiration and or regenerative cellular pathways and regenerative signaling.

### BACKGROUND

Regenerative medicine has made significant strides in recent years, with the discovery of various peptides, peptide bioregulators, and compounds that promote tissue repair, wound healing, and cellular regeneration. These agents exert their effects by modulating inflammatory responses, stimulating angiogenesis, promoting cell proliferation and migration, and enhancing cellular respiration. However, the efficacy of these regenerative agents is often limited by their short half-lives, ranging from minutes to a few hours, which necessitates frequent administration and limits their therapeutic potential.

### SUMMARY OF THE DISCLOSURE

The present disclosure addresses the limitations of existing regenerative therapies by providing a composition comprising a combination of (a) NAD+ and (b) one or more secondary active agents, wherein the one or more secondary active agents are selected from (i) therapeutic peptides, (ii) peptide bioregulators, and/or (iii) compounds that are believed to facilitate cellular respiration and/or regenerative cellular pathways (e.g., cellular respiration enhancers) and cellular regenerative pathways.

Applicant has unexpectedly demonstrated, and as described in the example set forth herein, that co-delivery of NAD⁺ with one or more secondary active agents selected from (i) therapeutic peptides, (ii) peptide bioregulators, and/or (iii) compounds that are believed to facilitate cellular respiration and or regenerative cellular pathways yields synergistic therapeutic outcomes in tissue injuries marked by mitochondrial dysfunction, energy depletion, and impaired repair signaling, particularly in ischemic contexts like myocardial infarction or chronic non-healing wounds.

By providing sustained transdermal NAD⁺ via anodal iontophoresis in tandem with one or more secondary active agents selected from (i) therapeutic peptides, (ii) peptide bioregulators, and/or (iii) compounds that are believed to facilitate cellular respiration and/or regenerative cellular pathways, the present disclosure uniquely addresses both energetic deficits and reparative blockade fidelity (Poljšak et al., 2023).

An open-label, parallel-group human study (see the Example, herein) further substantiates the synergistic anti-inflammatory action of the components included within the disclosed compositions. For instance, and as described further herein, in 120 adults with baseline hs-CRP≥5 mg L⁻¹, a single 12-h iontophoretic patch co-delivering NAD⁺ (500 mg) and KPV (10 mg) drove a mean absolute Δhs-CRP of −9.82±1.26 mg L⁻¹(93% reduction) by Day 7, significantly exceeding the declines observed with NAD⁺ (84%) or KPV (74%) monotherapy and exhibiting a markedly steeper descent within the first 24 h (Group×Day χ²=102.4, p<10⁻¹⁵). No serious adverse events were reported, confirming clinical tolerability while demonstrating a pharmacodynamic synergy that materially advances the disclosed compositions' therapeutic utility

A first aspect of the present disclosure is directed to the sustained, co-localized delivery of NAD⁺ and one or more secondary active agents, wherein the one or more secondary active agents are selected from (i) therapeutic peptides, (ii) peptide bioregulators, and/or (iii) compounds that are believed to facilitate cellular respiration and/or regenerative cellular pathways over a predetermined time period, such as a time period ranging from about 4 hours to about 16 hours, thereby bypassing hepatic first-pass metabolism and circumventing the inefficiencies of precursor-only approaches (e.g., NMN, NR) that rely on intracellular enzymatic conversion and which may not rapidly restore mitochondrial NAD⁺ under acute injury. This co-delivery modality ensures continuous energetic support for peptide-mediated signaling, enabling enhanced infarct-zone angiogenesis, accelerated wound granulation, and improved functional recovery metrics, potentially reducing infarct size or non-healing wound burden by between about 25 to about 35% relative to monotherapy regimens.

A second aspect of the present disclosure provides a composition comprising a combination of (a) NAD+, and (b) one or more secondary active agents, wherein one or more secondary active agents are compatible with transdermal delivery, such as iontophoretic delivery, while providing minimal skin irritation. “Iontophoresis” is defined as the use of electric current to drive molecules across cell membranes through an electrolyte solution. In a therapeutic context, iontophoresis or iontophoretic delivery is used to facilitate the administration of bioactive substances, either systemically or locally. In some embodiments, the one or more secondary active agents are selected from therapeutic peptides, peptide bioregulators, and/or compounds that are believed to facilitate cellular respiration and or regenerative cellular pathways. In some embodiments, the one or more one or more secondary active agents may include, but are not limited to, charged small molecules, peptides, or biologics that benefit from improved mitochondrial function or energy metabolism, such as anti-inflammatory agents, analgesics, antioxidants, or growth factors, provided they possess a net charge under optimized pH conditions suitable for iontophoretic transport. In some embodiments, the presently disclosed compositions have a pH ranging from about 4.8 to about 5.5 to facilitate anodic delivery through an iontophoretic device. In other embodiments, the compositions have a pH ranging from between about 7.5 to about 8.0 to facilitate cathodic delivery through an iontophoretic device. In some embodiments, the inclusion of such the one or more secondary active agents leverages the synergistic enhancement of cellular respiration provided by NAD+, thereby facilitating a broader range of regenerative or therapeutic outcomes when delivered transdermally via an iontophoretic system.

A third aspect of the present disclosure is an iontophoretic delivery system including a composition comprising a combination of (a) NAD+, and (b) one or more secondary active agents selected from (i) therapeutic peptides, (ii) peptide bioregulators, and/or (iii) compounds that are believed to facilitate cellular respiration and or regenerative cellular pathways. Wishing to be bound by any particular theory, it is believed that the presently disclosed delivery system enhances and/or prolongs the regenerative effects of either of the NAD+ agent and/or the one or more secondary active agents by providing a sustained delivery system, thereby optimizing cellular respiration and/or promoting tissue repair and regeneration.

A fourth aspect of the present disclosure is a system for iontophoretic transdermal delivery including an iontophoretic device comprising at least one agent reservoir adapted for storing a composition including (a) NAD+, and (b) one or more secondary active agents, wherein the one or more secondary active agents are selected from (i) therapeutic peptides, (ii) peptide bioregulators, and/or (iii) compounds that are believed to facilitate cellular respiration and or regenerative cellular pathways (e.g., cellular respiration enhancers). In some embodiments, the composition (such the composition to be stored in the reservoir) has a pH ranging from about 4.8 to about 5.5 to facilitate anodic delivery through an iontophoretic device. In other embodiments, the compositions have a pH ranging from between about 7.5 to about 8.0 to facilitate cathodic delivery through an iontophoretic device. Suitable iontophoretic devices and components of such iontophoretic devices include those described in U.S. Publication Nos. 20050070840, 20070066932, and 20090221985; and U.S. Pat. Nos. 7,945,320 and 9,492,650 the disclosures of which are hereby incorporated by reference herein in their entireties.

A fifth aspect of present disclosure is a kit comprising (i) a composition comprising a combination of (a) NAD⁺, and (b) one or more secondary active agents, wherein the one or more secondary active agents are selected from (i) therapeutic peptides, (ii) peptide bioregulators, and/or (iii) compounds that facilitate cellular respiration and or regenerative cellular pathways; and (ii) an iontophoretic delivery device. In some embodiments, the composition including the NAD+ and the one or more secondary regents is in lyophilized form. In some embodiments, the kit further comprises a predetermine amount of water, such as distilled deionized water. In some embodiments, the iontophoretic delivery device comprises a reservoir for storing the composition, at least one electrode, and an electrical energy source. In some embodiments, the iontophoretic delivery device comprises a reservoir for storing the composition, at least two electrodes, and an electrical energy source. In other embodiments, the device includes at least two electrodes and an integrated power module, all in a self-contained, wearable format. Suitable iontophoretic delivery devices and components of such iontophoretic delivery devices include those described in U.S. Publication Nos. 20050070840, 20070066932, and 20090221985; and U.S. Pat. Nos. 7,945,320 and 9,492,650 the disclosures of which are hereby incorporated by reference herein in their entireties. In some embodiments, the kit further includes a microneedling device.

A sixth aspect of the present disclosure is an iontophoresis patch system for the transdermal co-delivery of NAD⁺ and one or more secondary active agents, wherein the one or more secondary active agents are selected from (i) therapeutic peptides, (ii) peptide bioregulators, and/or (iii) compounds that facilitate cellular respiration and or regenerative cellular pathways (e.g., peptides such as BPC-157 or KPV). In some embodiments, the iontophoresis patch system incorporates both an anode and a cathode, wherein each of the anode and cathode are pre-coated with the appropriate electrode material and, wherein the patch includes a built-in galvanic power source that activates upon hydration. In some embodiments, delivery from the iontophoresis patch system proceeds via low-intensity direct current (DC) over approximately 14 hours, as exemplified by systems described in U.S. Pat. Nos. 6,653,014 B2 and 6,745,071 B, the disclosures of which are hereby incorporated by reference herein in their entireties. Such iontophoresis patch system iontophoresis patch system facilitate consistent current density (e.g., 0.05-0.1 mA/cm²), obviating external wiring and/or batteries. In some embodiments, the iontophoresis patch system is the IontoPatch® platform (available from IontoPatch, St. Paul, MN).

In some embodiments, the iontophoresis patch system includes: (a) one or more electrodes: a zinc anode and a silver/silver-chloride cathode for stable ion generation (U.S. Pat. Nos. 6,421,561; 6,653,014; 6,745,071; 7,016,723; 7,031,768; 7,031,769); (b) one or more drug reservoirs: a hydrated hydrogel matrix pre-loaded with the therapeutic formulation, positioned in contact with the appropriate electrode according to drug polarity; and/or (c) a power source: an embedded galvanic cell that initiates upon skin contact, generating a controlled DC current for the patch's lifetime. Adhesive and Backing Layers: biocompatible adhesive for secure placement and a flexible, moisture-resistant backing to protect internal components. In some embodiments, the iontophoresis patch system is the ActivaPatch® system (ActivaTek Inc.). The ActivaPatch® system offers both single-use and reusable configurations:
Electrodes: zinc anode and silver/silver-chloride cathode optimized for ionic conductivity (U.S. Pat. Nos. 6,775,570; 7,047,069; 8,197,844).
Drug Reservoir: disposable pre-filled gel cartridges (single-use) or replaceable drug modules (reusable) containing ionic therapeutics.
Power Source: in single-use models, a self-contained galvanic cell; in reusable models, a detachable battery-powered controller delivering adjustable DC current (up to 4 mA) with safety shut-off on dose completion.
Adhesive and Backing: medical-grade adhesive interfaces and robust, water-resistant backing for both disposability and multiple-use reliability

While IontoPatch® and ActivaPatch® illustrate embodiments of single-use and reusable iontophoretic devices, the scope of the disclosure encompasses any iontophoresis platform, whether fully integrated patches, modular systems, or hybrid designs, that employs comparable electrode configurations, hydration-activated power sources, and controlled DC delivery profiles for the co-delivery of NAD⁺ or its precursors in combination with one or more therapeutic peptides, peptide bioregulators, or compounds that are believed to facilitate cellular respiration and or regenerative cellular pathways and regenerative signaling.

A seventh aspect of the present disclosure is a kit comprising (i) a composition comprising a combination of (a) NAD+ and (b) one or more secondary active agents, wherein the one or more secondary active agents are selected from (i) therapeutic peptides, (ii) peptide bioregulators, and/or (iii) compounds that are believed to facilitate cellular respiration and or regenerative cellular pathways; and (ii) an iontophoretic delivery circuit. In some embodiments, the iontophoretic delivery circuit comprises at least two electrodes and an electrical energy source (e.g., a battery). In some embodiments, the iontophoretic delivery circuit further comprises a reservoir for holding the composition (such as after the composition has been reconstituted).

An eighth aspect of the present disclosure is iontophoresis device, including (a) an active agent reservoir that stores a quantity of a composition, wherein the composition includes NAD+ and one or more secondary active agents, wherein the one or more secondary active agents are selected from (i) therapeutic peptides, (ii) peptide bioregulators, and/or (iii) compounds that are believed to facilitate cellular respiration and or regenerative cellular pathways; and (b) an active electrode element operable to apply an electrical potential of a first polarity to deliver at least a portion of the composition to a biological interface from the iontophoresis device. In some embodiments, the biological interface is skin, e.g., the skin of a human/mammalian subject. In some embodiments, the composition includes KPV tripeptide. In some embodiments, the composition further includes sodium citrate.

A ninth aspect of the present disclosure is a kit comprising (a) a transdermal delivery device; and (b) a composition comprising NAD+ and one or more secondary active agents. In some embodiments, the transdermal delivery device is an iontophoretic delivery device. In some embodiments, the iontophoretic delivery device comprises a reservoir, at least one electrode, and a power source. In some embodiments, the one or more secondary active agents are therapeutic peptides or peptide bioregulators. In some embodiments, the composition further comprises sodium citrate. In some embodiments, the composition comprises NAD+ and KPV tripeptide. In some embodiments, the composition further comprises sodium citrate. In some embodiments, the pH is about 5. In some embodiments, the kit further comprises water. In some embodiments, the composition is within a first container; and wherein the water is in a second container. In some embodiments, the kit further comprises a microneedling device.

A tenth aspect of the present disclosure is a kit configured to support the administration of NAD⁺-based formulations using an iontophoretic delivery platform. In some embodiments, the kit comprises multiple elements assembled to facilitate consistent preparation, activation, and application of a transdermal treatment cycle in a clinical or home-use environment. In some embodiments, the kit may include a plurality of iontophoretic patches, for example six (6) single-use ActivaPatch® IontoGo™ 12.0 devices, each configured as described herein with an integrated reservoir (e.g., a hydrogel drug reservoir), one or more active and/or counter electrodes, an adhesive substrate, and a self-contained power source dimensioned to deliver a dose of the compositions disclosed herein, each to deliver a dose of approximately 80 milliampere-minutes over a predetermined time period, such as a twelve-hour period. In some embodiments, each patch is individually packaged in a protective sterile barrier pouch to preserve integrity prior to use. In some embodiments, kit further comprises six (6) amber glass vials with screw-top closures. In some embodiments, each vial contains a premeasured amount of blended NAD⁺ formulation, optionally in combination with adjunctive compounds such as glutathione, peptide bioregulators, or stabilizing buffers. In some embodiments, the amber glass construction minimizes photodegradation of the formulation, while the threaded cap ensures resealing after initial opening. In some embodiments, the vials are dimensioned for single-use reconstitution to avoid contamination and preserve purity. In some embodiments, kit further comprises six (6) sterile water ampoules, each individually sealed and configured to provide a measured diluent volume suitable for reconstitution of the contents of the amber vials. Upon reconstitution, the solution is applied directly to the hydrogel drug reservoir of the patch prior to application to the skin. The provision of matched diluent ampoules ensures consistent formulation strength and pH across all treatment cycles. In some embodiments, kit additionally includes a microneedling device configured for dermatological pretreatment of the skin at the intended application site. In some embodiments, the microneedling device may be provided in a variety of operational formats, including roller-type heads, linear stamp-type arrays, and oscillating pen-style applicators. In some embodiments, each format incorporates a plurality of fine needles, typically ranging from 0.25 to 1.0 millimeters in length, sufficient to create transient microchannels in the stratum corneum without inducing bleeding or long-term disruption of skin architecture. In some embodiments, the microneedling is applied to an area approximately equal in size to the hydrogel reservoir of the iontophoretic patch, thereby localizing the effect to the precise region of intended drug delivery. In some embodiments, the procedure is carried out immediately prior to placement of the patch, ensuring that the transient microchannels remain patent during the initial period of iontophoretic transport. In some embodiments, the microneedling step provides distinct mechanistic advantages in this specific application. First, by physically breaching the outer stratum corneum, microneedling reduces the electrical impedance of the skin, thereby lowering resistance across the delivery site. This reduction in impedance allows the iontophoretic current to flow more efficiently, minimizing energy loss and improving dose accuracy. Second, the transient microchannels formed by microneedling create aqueous pathways through which both charged and neutral molecules may more readily diffuse. This structural modification enhances electromigration of ionic species, such as NAD⁺, and simultaneously augments electroosmotic solvent flow, which supports the transport of neutral co-formulants such as glutathione. Third, the mechanical stimulation associated with microneedling is known to trigger a localized biological response, including transient vasodilation and upregulation of repair pathways, which may further improve systemic uptake and tolerance of the delivered compounds.

In some embodiments, the combination of microneedling pretreatment and iontophoretic delivery provides a synergistic mechanism by which both barrier reduction and active electromotive driving forces act in concert to achieve enhanced transdermal flux. In some embodiments, the microneedling ensures initial pathway formation across the high-resistance epidermal barrier, while the iontophoresis maintains a sustained electromotive gradient to drive penetration of molecules that otherwise would not achieve sufficient permeability. In some embodiments, this dual-action approach is particularly advantageous for bioactive compounds such as NAD⁺ and therapeutic peptides, whose molecular size, polarity, or hydrophilicity limit their passive diffusion across the intact stratum corneum.

In some embodiments, the kit configuration described herein facilitates a standardized treatment protocol comprising (1) reconstitution of the NAD⁺ formulation using the supplied sterile water ampoule, (2) loading of the solution into the hydrogel reservoir of a single-use patch, (3) pretreatment of the application site by microneedling in a roller, stamp, or oscillating pattern across an area approximately equal to the reservoir footprint immediately prior to patch placement, and (4) application of the patch for a sustained twelve-hour iontophoretic delivery cycle. By integrating six complete treatment sets within a single packaged system, the kit provides a controlled, repeatable, and clinically optimized regimen for the systemic delivery of NAD⁺ formulations and related compounds.

An eleventh aspect of the present disclosure is a method of enhancing transdermal delivery of one of the compositions of the present disclosure, comprising: (a) creating a plurality of microchannels in skin by microneedling; (b) applying an iontophoretic device to the microchanneled skin within a predefined defined time window after microneedling; and (c) applying electrical current to drive the active agent through the skin, wherein a delivery flux is greater than iontophoresis alone. In some embodiments, the one or more compositions include NAD+ and one or more secondary active agents, wherein the one or more secondary active agents are selected from (i) therapeutic peptides, (ii) peptide bioregulators, and/or (iii) compounds that are believed to facilitate cellular respiration and or regenerative cellular pathways. In some embodiments, the one or more secondary active agents are selected glutathione, GHK-Cu, KPV, thymosin-β4 and/or TB-500 fragments.

In some embodiments, the method comprises initiating iontophoresis within about 10 to about 60 minutes of microneedling. In some embodiments, the method comprises initiating iontophoresis within about 10 to about 40 minutes of microneedling. In some embodiments, the method comprises initiating iontophoresis within about 10 to about 30 minutes of microneedling. In some embodiments, the method comprises initiating iontophoresis within about 10 minutes of microneedling. In some embodiments, the method comprises initiating iontophoresis within about 20 minutes of microneedling. In some embodiments, the method comprises initiating iontophoresis within about 30 minutes of microneedling.

In some embodiments, the electrical current density is 0.01-0.5 mA/cm², such as for a time period ranging from about 5 to about 360 minutes. In some embodiments, the iontophoretic delivery after microneedling achieves a ≥5× flux increase compared to iontophoresis without microneedling. In some embodiments, the plurality of microchannels each have depth ranging from about 150 to about 800 μm, a diameter from between about 20 to about 200 μm, and a density ranging from between about 100 to about 1,200 channels/cm². In some embodiments, the method further comprises measuring impedance and adjusting current to maintain cumulative charge between about 1 to about 100 mC/cm². In some embodiments, a composition introduced to a reservoir of the iontophoretic device is buffered to a pH ranging from between about 5.2 to about 7.4. In some embodiments, a composition introduced to a reservoir of the iontophoretic device has an osmolality ranging from between about 200 to about 400 mOsm/kg. In some embodiments, a composition introduced to a reservoir of the iontophoretic device includes a stabilizer selected from trehalose, mannitol, and/or glycerol. In some embodiments, a reservoir of the iontophoretic device includes cross-linked hydrogel matrices having a conductivity ranging from between about 10 to about 30 mS/cm.

A twelfth aspect of the present disclosure is a system comprising: (i) a microneedling device; (ii) an iontophoretic patch including an electrode and reservoir containing an active agent; and (iii) a controller that adjusts current during delivery, wherein the system is configured for sequential or concurrent microneedling and iontophoresis. In some embodiments, the system further comprises a composition including NAD+ and one or more secondary active agents, wherein the one or more secondary active agents are selected from (i) therapeutic peptides, (ii) peptide bioregulators, and/or (iii) compounds that are believed to facilitate cellular respiration and or regenerative cellular pathways.

A thirteenth aspect of the present disclosure is a kit comprising: (a) a sterile microneedle array; (b) an iontophoretic patch having a reservoir containing an active agent; and (c) instructions directing microneedling followed by iontophoresis, wherein delivery is initiated within a predetermined time after microneedling.

A fourteenth aspect of the present disclosure is a kit comprising: (a) a sterile microneedle array; (b) an iontophoretic patch having a reservoir; and (c) a composition including NAD+ and one or more secondary active agents, wherein the one or more secondary active agents are selected from (i) therapeutic peptides, (ii) peptide bioregulators, and/or (iii) compounds that are believed to facilitate cellular respiration and or regenerative cellular pathways

### BRIEF DESCRIPTION OF THE FIGURES

FIG. 1 demonstrates a mean CRP reduction over the study period. In particular, this plot shows the mean CRP for each treatment arm at the start of the study (Baseline, Day 1) and after one week (Day 7).

FIG. 2 shows the distributional shift in CRP from baseline to Day 7 across treatment arms. Box-and-whisker plots display individual serum CRP values (mg L⁻¹) for each treatment arm at study entry and after 7 days. Baseline medians were comparable. Combo 9.8, NAD⁺9.9, KPV 9.6 mg L⁻¹ confirming randomization balance. By Day 7 the Combo median fell to 0.68 mg L⁻¹ (IQR 0.35-1.1); NAD⁺ and KPV medians were 1.55 mg L⁻¹ (0.9-2.6) and 2.45 mg L⁻¹ (1.7-3.2), respectively. Two-sided Wilcoxon matched-pairs tests showed highly significant within-arm reductions (P<0.001). A Kruskal-Wallis test indicated an overall difference on Day 7 (P<0.001); Dunn's post-hoc comparisons confirmed the Combo arm was lower than both monotherapies (q<0.01, FDR-adjusted). Boxes denote the inter-quartile range, horizontal lines the medians, whiskers extend to 1.5× IQR, and open circles mark outliers. The dashed vertical line separates baseline from endpoint measurements.

FIG. 3 shows the magnitude of per-subject CRP reduction after 7 days of treatment. Box-and-whisker plots show per-subject ΔCRP (baseline−Day 7) for the combination patch (n=40), NAD⁺ patch (n=40) and KPV patch (n=38). Medians (horizontal lines) were 10.1, 8.1 and 6.6 mg L⁻¹, respectively; boxes span the inter-quartile range (IQR), whiskers extend to 1.5×IQR, and open circles mark outliers. A Kruskal-Wallis test demonstrated an overall group effect (H=22.3, P<0.001). Two-sided Mann-Whitney U post-hoc tests (Holm corrected) yielded ***P<0.001 for Combo vs NAD⁺, **P<0.01 for Combo vs KPV, and *P=0.034 for NAD⁺ vs KPV (significance codes are placed above the corresponding brackets).

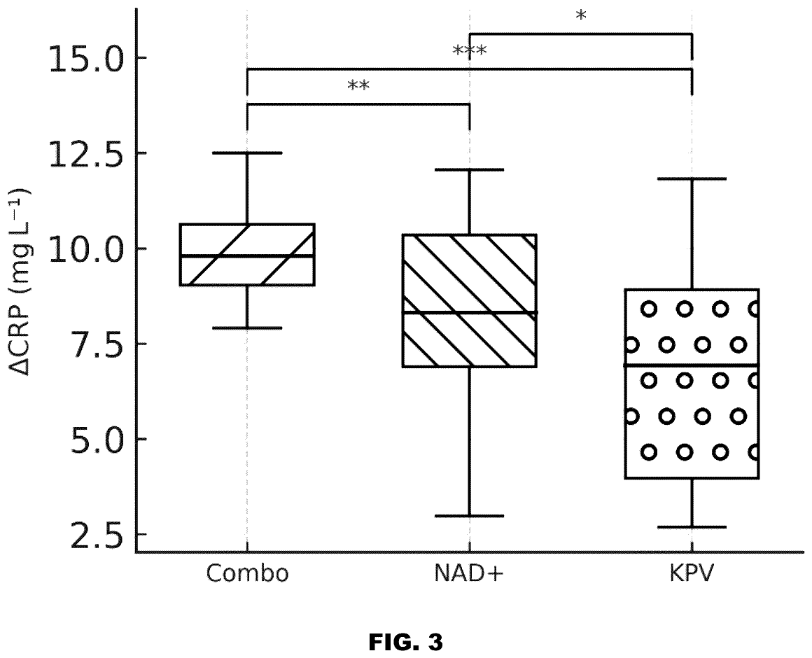

FIG. 4 provides the percentage reduction in CRP after 7 days of therapy. Bars show the mean±SEM percentage drop in serum CRP for each treatment arm, with every participant's value plotted as a jittered black×to illustrate inter-individual variability. Distinct hatching facilitates black-and-white reproduction. Combined NAD⁺+KPV: mean 94% (median 94%, n=40). NAD⁺ only: mean 83% (median 83%, n=40). KPV only: mean 72% (median 74%, n=40). A Kruskal-Wallis test indicated a significant overall group effect (H=28.2, P<0.001). Pair-wise two-sided Mann-Whitney U tests (brackets above bars) showed the combination arm achieved a significantly greater CRP reduction than either monotherapy (**P<0.001 for both comparisons), and NAD⁺ outperformed KPV alone (**P<0.001). The tight clustering of points around the 90-100% range in the combination group highlights both the magnitude and consistency of the response.

FIG. 5 demonstrates that the inflammatory burden (AUC) is lowest with combined NAD⁺+KPV therapy. Box-and-whisker plots depict the area under the CRP concentration-time curve from Day 1 to Day 7 (AUC₁₋₇, mg L⁻¹·day) for each treatment arm. Median AUC values were 28 mg L⁻¹·day (Combo), 55 mg L⁻¹·day (NAD⁺), and 40 mg L⁻¹·day (KPV), indicating that the combination regimen halved cumulative inflammatory exposure relative to NAD⁺ alone and cut it by ˜30% versus KPV. Overall differences were significant (Kruskal-Wallis H=31.4, P<0.001). Pair-wise Mann-Whitney tests confirmed lower AUC in the Combo arm than either monotherapy (**P<0.001 for both) and a significant advantage of NAD⁺ over KPV (**P<0.001). Boxes show IQRs, central lines mark medians, whiskers extend to 1.5×IQR, and open circles denote outliers.

FIG. 6 illustrates the CRP response profiles within each treatment arm. Waterfall bars represent the percentage reduction in CRP for every participant, ranked from best to least responder within each arm and displayed in three separate blocks (Combo, NAD⁺, KPV; hatching as in legend). Uniform high responders: All Combo subjects achieved ≥85% reduction, clustering tightly near 100%. Variable responders: NAD⁺ and KPV monotherapy arms show broader distributions, tapering down to 50% and 30% reductions, respectively. Minimal overlap: The right edge of the Combo block (≈82%) still exceeds the median responses in both monotherapy blocks, underscoring the superior and consistent efficacy of the combined regimen.

FIG. 7 illustrates that the combination patch outperforms the Bliss-additive expectation for cumulative inflammatory load.

FIG. 8 illustrates a representative plan view of an iontophoretic patch device according to certain embodiments of the present disclosure. The patch comprises a generally bilobed configuration in which the upper circular portion accommodates a hydrogel reservoir, while the lower portion houses a counter electrode assembly. The hydrogel reservoir is positioned centrally within the circular portion and is dimensioned to receive a therapeutic formulation, for example an NAD⁺ solution or peptide blend, immediately prior to use. The reservoir is in direct ionic communication with an underlying delivery electrode, such that upon activation, charged species are driven across the skin in alignment with the electrode polarity. The device further includes an adhesive backing layer extending across the full surface of the patch, which secures the patch to the skin and provides occlusion throughout the delivery cycle. An activation tab is disposed at the intersection of the circular and lower portions of the device, permitting initiation of the embedded power source, such as a galvanic cell, at the time of application. The lower portion of the patch contains a gel block electrode that functions as a counter electrode, completing the circuit during iontophoretic operation.

FIG. 9 provides a cross-sectional schematic view of the iontophoretic patch device shown in FIG. 10, illustrating the layered structure and operative components. The hydrogel reservoir is positioned at the upper surface of the patch and overlies the delivery electrode, which in certain embodiments may comprise a silver/silver chloride or zinc-based conductor. A galvanic electrode is positioned in the lower portion of the device and is supported by an associated gel block to ensure skin contact and ionic continuity. The system is powered by an embedded zinc-air cell, represented centrally within the device, which generates a constant low-level current over the intended twelve-hour delivery period. A microcontroller may be positioned adjacent the power source to regulate current output, ensuring the programmed dose of approximately 80 milliampere-minutes is delivered reliably and within therapeutic safety limits. The entire assembly is covered by a flexible backing layer that provides mechanical support and insulation, while maintaining biocompatibility and conformability for skin adhesion.

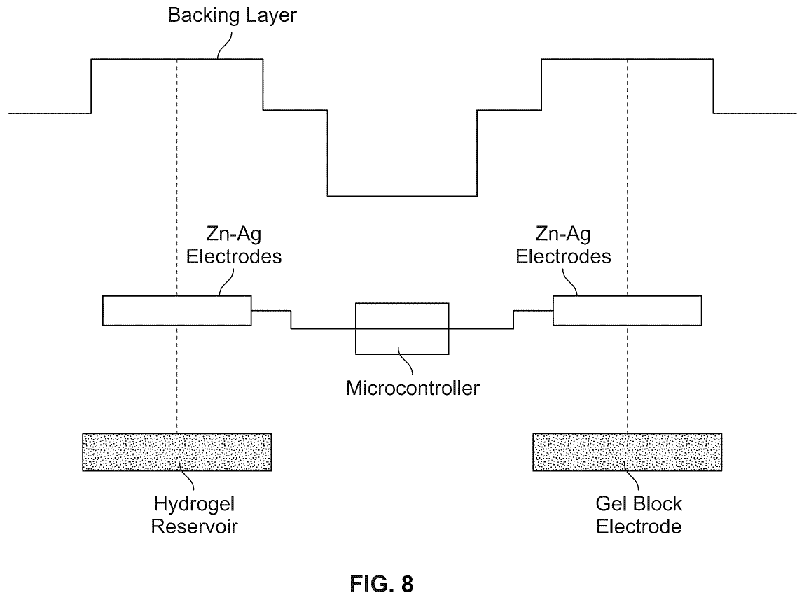

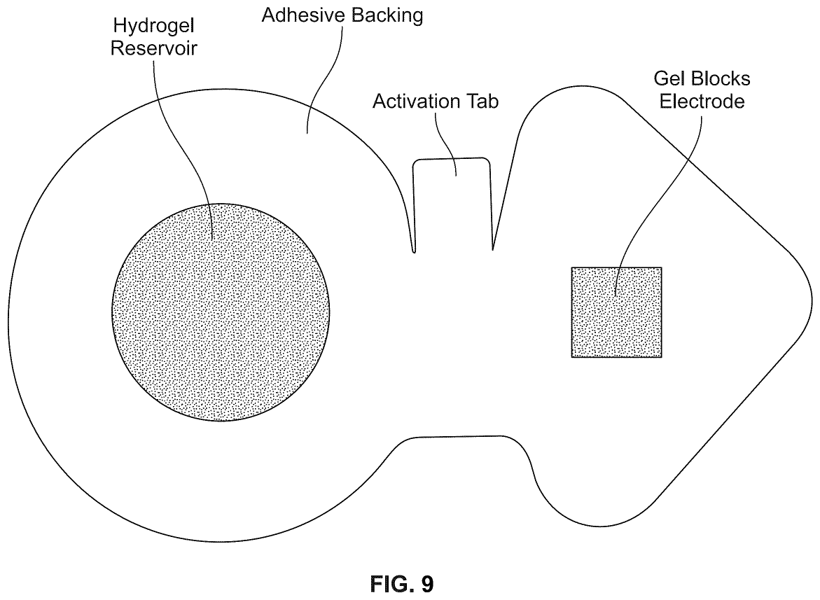

Together, FIGS. 8 and 9 illustrate the spatial configuration and internal architecture of the iontophoretic patch system, including the hydrogel reservoir, activation tab, adhesive backing, delivery and counter electrodes, galvanic power source, and regulatory microcircuitry, thereby enabling a compact, self-contained, single-use transdermal delivery device.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of,” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### Overview

A primary difficulty in employing iontophoresis for delivering a composition including a combination of NAD+ and one or more secondary active agents (e.g., one or more (i) therapeutic peptides, (ii) peptide bioregulators, and/or (iii) compounds that are believed to facilitate cellular respiration and or regenerative cellular pathways or initiate regenerative cellular signaling) lies in the pH-dependent charge behavior of the constituent molecules, which directly affects their transport efficiency.

Iontophoresis involves the use of low-intensity current (usually <0.5 mA/cm²) to transport both charged and neutral species into and through the skin, based on two main mechanisms of action: electromigration and electro-osmosis. Electromigration is based on the principle that like charges repel each other. As such, iontophoresis relies on the principle of like charges repelling, positively charged molecules are driven from the anode (positive electrode), while negatively charged molecules are driven from the cathode (negative electrode).

During electromigration, charged molecules move under the influence of an electrical field, when in contact with an electrode of the same charge. Hence, when the electrical current is applied, cations are repelled by the anode and anions by the cathode, moving into the skin. In electro-osmosis, a bulk flow of fluid, also called solvent flow, is driven by a difference in electrical potential across a charged, porous membrane. The electrical field causes the free counter-ions of the charged membrane to migrate towards the oppositely charged electrode, carrying water molecules in the process. This results in a solvent flow that, in turn, carries neutral as well as charged molecules along with it, in the same direction. The direction of the flow depends on the charge in the biological membrane.

By way of example, NAD+ and its precursors, such as nicotinamide riboside (NR), exhibit variable charge states depending on pH. NR is neutral at a physiological pH of 7; but becomes positively charged at a pH of 5 due to protonation of its pyridine ring. Therapeutic peptides, such as BPC-157 or GHK-Cu, similarly possess pH-dependent charges influenced by their amino acid compositions, with net charges shifting from positive to neutral or negative as pH increases across their isoelectric points. Combining these “active” agents (i.e., NAD+ and BPC-157 or GHK-Cu) in a single formulation requires a pH that simultaneously maintains the appropriate charge state for each “active” component of the composition to enable effective iontophoretic transport from the same electrode, a task complicated by their differing chemical properties and optimal pH ranges.

This pH optimization is further constrained by the need to minimize skin irritation, a known limitation of iontophoretic systems. Excessively acidic (e.g., pH 5) or alkaline conditions can cause substantial skin irritation or burns, rendering the delivery system unacceptable to users. For instance, delivering NR at pH 5 to achieve a positive charge might enhance its transport from the anode but could irritate the skin, while a neutral pH of 7, safer for skin contact, renders NR uncharged and thus incompatible with iontophoresis. For instance, peptides like KPV, which may most effectively undergo, could conflict with NAD+'s requirements.

When formulating iontophoretic systems that co-deliver both NAD⁺ and BPC-157, the complexity of optimizing electrokinetic transport is significantly magnified due to the divergent charge architectures and transport mechanisms of the two compounds. For instance, NAD⁺, a highly anionic molecule under physiological conditions, typically carries a net charge between about 2 and about 3 at pH values compatible with skin tolerability (pH 5.0-7.0), driven primarily by its pyrophosphate moieties. In contrast, BPC-157 exhibits a distributed charge landscape, composed of both cationic (N-terminal and lysine) and anionic (glutamic acid, aspartic acid, and C-terminal) residues, resulting in a net charge of approximately 0.5 at pH 5.0. The differing electrochemical behavior of these two molecules necessitates precise control over pH, buffer composition, and ionic strength to ensure simultaneous and directional transport from a shared electrode interface.

This dual-agent configuration introduces a formulation constraint that must harmonize the competing requirements for electrophoretic migration and electroosmotic facilitation. While NAD⁺, being strongly anionic, relies primarily on cathodal delivery or passive electroosmotic drag from the anode, BPC-157's partial compatibility with anodal delivery is mediated by its cationic subunits and net near-neutral charge at mildly acidic pH. Accordingly, a shared anodal delivery route is theoretically feasible but contingent on achieving a formulation pH that sufficiently protonates the phosphate groups of NAD⁺ to reduce net negative charge, while maintaining BPC-157 in a partially protonated state that preserves the positive charge on the lysine and N-terminal amine.

Moreover, the differential ion mobility of NAD⁺ and BPC-157 across the stratum corneum further complicates formulation design. The smaller molecular weight and higher charge density of NAD⁺ favor rapid migration under an electric field but may lead to competitive inhibition of BPC-157 transport due to ionic crowding and current partitioning. Conversely, excess buffering to suppress local pH shifts and reduce skin irritation, while beneficial from a tolerability standpoint, may attenuate the electrophoretic potential gradient necessary to drive either molecule effectively. These factors demand a carefully tuned formulation matrix that not only balances the subunit and net charge states of each therapeutic compound but also integrates skin-compatible pH and ionic environment optimization.

Therefore, co-formulation of NAD⁺ and BPC-157 for iontophoretic delivery represents a nontrivial electrochemical engineering problem wherein pH-dependent charge states, subunit-specific ionic interactions, and the interplay between electrophoresis and electroosmosis must be harmonized. The formulation must be sufficiently acidic to support partial protonation of NAD⁺ phosphate groups and preserve the cationic domains of BPC-157 yet buffered to remain within dermatologically acceptable tolerability thresholds. Such optimization strategies necessitate empirical titration of buffer systems, electrode polarity configuration, and total ionic strength to enable concurrent delivery with maximal bioavailability and minimal dermal irritation.

In the context of iontophoretic delivery, peptides such as BPC-157 exhibit complex charge behavior that must be accounted for in both formulation design and electrode selection. Although the molecule as a whole possesses a net charge, approximately −0.5 at pH 5.0, this aggregate value does not fully describe the electrokinetic behavior of the molecule under an applied current. Rather, BPC-157 comprises individual amino acid subunits, each of which maintains distinct acid-base equilibria and contributes independent ionic character to the overall molecule. Specifically, the side chains of glutamic acid (Glu) and aspartic acid (Asp), along with the C-terminal carboxyl group, retain negative partial charges at physiologically relevant pH values, including pH 5.0. These localized anionic centers contribute to the molecule's interaction with the electric field and may facilitate partial attraction to the anode through electroosmotic flow and localized electrophoretic interaction.

Conversely, the N-terminal amine and the s-amino group of the lysine (Lys) side chain are both positively charged at pH 5.0, each contributing a discrete+1 charge. These cationic regions establish localized domains of positive electrostatic potential that enhance compatibility with anodal migration. As a result, BPC-157's electrotransport behavior under iontophoresis is not solely dictated by its net molecular charge, but rather by a distributed charge architecture wherein distinct subunits exhibit opposing ionic tendencies. This charge heterogeneity enables complex interactions with both the applied electric field and the surrounding electrochemical environment, allowing partial alignment with anodal delivery despite a marginally negative overall charge.

The capacity for BPC-157 to undergo effective anodal transport is therefore driven by a dynamic interplay between electrophoretic mobility, electroosmotic drag, peptide conformation, and hydration shell dynamics. Electroosmosis, the solvent flux toward the cathode induced by the net cationic character of the skin, may co-transport neutrally or weakly negatively charged peptides such as BPC-157, especially when positively charged domains are present. Accordingly, the formulation must be optimized to exploit both charge distribution and solvent flow, allowing for enhanced transdermal transport from the anode despite an unfavorable net charge

This nuanced electrochemical profile underscores the necessity of formulating iontophoretic preparations in consideration of both whole-molecule net charge and the protonation states of individual amino acid residues. The Glu, Asp, and C-terminal moieties contribute negatively charged regions, while the N-terminal amine and lysine side chain contribute positively charged centers. The relative balance among these groups is highly pH-sensitive and determines the effective electrokinetic response of the molecule. Accordingly, buffer systems such as sodium citrate are employed to stabilize formulation pH and reduce skin irritation, but must be carefully balanced, as they alter ionic strength and may compete with the active agent for ionic mobility, thereby influencing both efficacy and tolerability of the delivery system

Additionally, identifying the proper electrode terminal (anode or cathode) for delivery introduces additional hurdles when combining NAD⁺ with one or more therapeutic compounds. In conventional single-agent systems, such as the patient-controlled fentanyl patch described in U.S. Pat. No. 5,697,896, which employs a single drug reservoir at one electrode and inert electrolyte at the other, only one ionic species is delivered, precluding concurrent administration of a second agent with opposing charge requirements. Similarly, U.S. Pat. No. 5,843,015 teaches modification of a single peptide to optimize its own iontophoretic transport but does not contemplate simultaneous transport of an additional therapeutic under a different polarity. Likewise, U.S. Pat. No. 4,383,529 utilizes a drug-loaded gel in the donor electrode and a plain electrolyte gel in the return electrode, with no provision for dual active reservoirs or charge-balancing strategies. Consequently, attempting to co-deliver NAD⁺ and a cationic peptide (e.g., GHK-Cu) alongside a neutral or anionic cofactor such as coenzyme Q10 would fall outside the scope of these single-agent designs and would necessitate complex dual-electrode or sequential delivery schemes incompatible with standard patch simplicity.

The interdependence of pH, charge, electrode selection, and stability create a multidimensional optimization problem that would not be readily apparent to the person of ordinary skill in the art. For instance, merely adjusting pH to favor NAD+ iontophoretic delivery might neutralize a peptide's charge, halting its transport; while buffering to protect skin might dilute the electric field's effectiveness, reducing overall delivery rates. The present disclosure overcomes the aforementioned formulation challenges through the development of a novel buffered iontophoretic composition specifically engineered to stabilize pH, minimize electrochemical irritation, and maintain directional transport efficacy for both NAD⁺ and BPC-157. Unlike prior art systems that treat single-agent delivery in isolation or disregard the pH-sensitivity of therapeutic peptides and cofactors, the disclosed formulation simultaneously accommodates the molecular charge complexities of both compounds across relevant physiological pH ranges. This is achieved by incorporating a carefully titrated buffering system, specifically, sodium citrate, into the formulation matrix, thereby mitigating the adverse electrochemical effects associated with prolonged iontophoretic application.

Although both NAD⁺ and BPC-157 contain structural subunits that contribute negatively charged domains under mildly acidic conditions (pH about 5.0 to about 6.0), primarily from the phosphate groups of NAD⁺ and the Glu, Asp, and C-terminal residues of BPC-157, anodal delivery was selected and shown to be effective. This counterintuitive polarity configuration leverages the physiologic principle of electroosmotic flow, a bulk solvent movement from the anode toward the cathode, which can co-transport neutral or weakly anionic molecules along with the solvent front. In the context of human skin, which exhibits net negative fixed charges in the stratum corneum, electroosmosis predominates over electrophoresis in many practical cases of transdermal delivery. As a result, despite the partial anionic nature of both therapeutic agents, effective anodal transport was achieved through the synergistic action of electroosmotic drag and partial electrophoretic compatibility, particularly in the case of BPC-157, whose lysine and N-terminal amine residues retain discrete+1 charges at the target pH.

Mechanistically, sodium citrate functions through multiple pathways to reduce skin irritation and stabilize iontophoretic conditions. First, as a triprotic weak acid with pKa values of about 3.1, about 4.8, and about 6.4, citrate provides effective buffering capacity within the about 4.5 to about 6.5 pH window, precisely the range in which both NAD⁺ and BPC-157 exhibit optimal transport behavior with minimal charge antagonism. By resisting pH excursions at the electrode-skin interface, citrate limits the accumulation of protons (at the anode) or hydroxide ions (at the cathode) that would otherwise disrupt epidermal barrier integrity and provoke nociceptive responses. In unbuffered systems, these pH shifts are known to trigger localized acidosis or alkalosis, leading to erythema, stinging, or full-thickness burns, especially during extended wear periods typical of regenerative patch therapies.

Second, citrate ions act as mild chelators of divalent cations (e.g., calcium and magnesium) in the stratum corneum, which may otherwise precipitate under low pH and contribute to localized osmotic stress and irritation. Chelation moderates the electrochemical microenvironment, indirectly reducing inflammatory mediator release and improving barrier tolerability. Importantly, the inclusion of citrate at controlled concentrations also avoids excessive ionic competition, thereby preserving the electrokinetic driving force for NAD⁺ and BPC-157 transport.

Third, the citrate buffer system contributes to ionic strength modulation without overwhelming the current-carrying capacity of the patch. This is critical in dual-delivery systems, where electrokinetic efficiency must be maintained despite differing ion mobility profiles and molecular sizes. The citrate ions' intermediate mobility supports stable current distribution, avoiding abrupt impedance changes that could cause uneven delivery or skin resistance spikes, both of which compromise therapeutic consistency and user comfort.

Collectively, these features address the inherent limitations of unbuffered iontophoresis systems and facilitate reproducible, irritation-minimized delivery of bioactive peptides and cofactors. The result is a stable, pH-balanced, electrochemically optimized delivery platform applicable to all compositions disclosed herein, including but not limited to NAD⁺, BPC-157, and their analogs or functional derivatives. Critically, the use of the anode as the delivery interface in this system reflects not only the charge compatibility of select subunits within each molecule, but also a deliberate exploitation of electroosmotic flow as a dominant transport mechanism, enabling effective delivery even for molecules with nominally unfavorable electrophoretic polarity. This integrated approach to electrokinetic design constitutes a significant advance over existing technologies by ensuring molecular compatibility, transport efficiency, and end-user tolerability within a single, unified patch system.

### Compositions

The present disclosure provides a composition comprising a combination of (a) NAD+, and (b) one or more secondary active agents, wherein the one or more secondary active agents are selected from (i) therapeutic peptides, (ii) peptide bioregulators, and/or (iii) compounds that are believed to facilitate cellular respiration and or regenerative cellular pathways. In some embodiments, the compositions of the present disclosure are formulated for delivery via an iontophoretic delivery device or iontophoretic system. While iontophoresis offers a promising approach for transdermal delivery by utilizing an electric field to drive charged molecules through the skin, combining NAD+ and the one or more secondary active agents in such a system presents significant technical challenges. By way of example, these challenges are derived from the need to balance pH, molecular charge states, skin tolerability, and/or electrode selection (anode or cathode), each of which must be carefully optimized to ensure effective delivery and practical utility, as detailed below.

The compositions of the present disclosure include NAD+. NAD+ is believed to play an important role in the synthesis of adenosine triphosphate (ATP), an organic compound that provides energy for many processes in living cells, such as muscle contraction, nerve impulse propagation, condensate dissolution, and chemical synthesis, and as such NAD+ is a crucial nutrient for animal health.

In some embodiments, an amount of NAD+ in any of the compositions disclosed herein ranges from about 8% to about 50% by total weight of the composition. In some embodiments, an amount of NAD+ in any of the compositions disclosed herein ranges from about 10% to about 50% by total weight of the composition. In some embodiments, an amount of NAD+ in any of the compositions disclosed herein ranges from about 10% to about 45% by total weight of the composition. In some embodiments, an amount of NAD+ in any of the compositions disclosed herein ranges from about 10% to about 40% by total weight of the composition. In some embodiments, an amount of NAD+ in any of the compositions disclosed herein ranges from about 10% to about 30% by total weight of the composition. In other embodiments, the amount of NAD+ in the composition ranges from about 14% to about 25% by total weight of the composition. In other embodiments, the amount of NAD+ in the composition ranges from about 14% to about 20% by total weight of the composition. In other embodiments, the amount of NAD+ in the composition ranges from about 20% to about 25% by total weight of the composition. In yet other embodiments, the amount of NAD+ in the composition is about 10%, such as about 12%, such as about 14%, such as about 16%, such as about 18%, such as about 20%, such as about 22%, such as about 24%, such as about 26%, such as about 28%, such as about 30%, etc.

The compositions of the present disclosure also include one or more secondary active agents, wherein the one or more secondary active agents are selected from (i) therapeutic peptides, (ii) peptide bioregulators, and/or (iii) compounds that are believed to facilitate cellular respiration and or regenerative cellular pathways (hereinafter referred to as a “second active agent”). In some embodiments, the one or more second active agents are each independently present in an amount ranging from between about 0.1 wt % to about 5.0 wt % by total weight of the composition. In some embodiments, the one or more second active agents are each independently present in an amount ranging from between about 0.2 wt % to about 3.0 wt % by total weight of the composition. In other embodiments, the one or more second active agents are each independently present in an amount ranging from between about 0.5 wt % to about 2.0 wt % by total weight of the composition. In yet other embodiments, the one or more second active agents are each independently present in an amount ranging from between about 0.5 wt % to about 1.0 wt % by total weight of the composition. In further embodiments, the one or more second active agents are each independently present in an amount ranging from between about 0.1 wt % to about 1.0 wt % by total weight of the composition.

Non-limiting examples of therapeutic peptides include:
ARA-290: Ac-CAEECRPIA-NH2 (denotes D-Ala)—Erythropoietin-derived peptide with tissue-protective and anti-inflammatory properties. (SEQ ID NO: 1)
BPC-157: GEPPPGKPADDAGLV—Body Protection Compound, promotes wound healing and tissue repair. (SEQ ID NO: 2)
B7-33: SLLGRMKGA—Immunomodulatory peptide derived from the CD80 protein. (SEQ ID NO: 3)
CJC-1295: YADAIFTNSYRKVLGQLSARKLLQDIMSR-OH—Growth hormone-releasing hormone (GHRH) analog with enhanced stability. (SEQ ID NO: 4)
DSIP: WAGGDASGE-Delta Sleep-Inducing Peptide, promotes sleep and has neuroprotective properties. (SEQ ID NO: 5)
FOX04-DRI: D-Arg-Lys-Leu-His-D-Ala-NH2—Senolytic peptide that selectively eliminates senescent cells. (SEQ ID NO: 6)
GHK-Cu: GHK-Cu (Copper complex)—Tripeptide with wound healing and tissue regeneration properties.
GHRP-2: H-D-Ala-D-2-Nal-Ala-Trp-D-Phe-Lys-NH2—Growth Hormone-Releasing Peptide-2, stimulates growth hormone secretion ((D2Nal)=D-2-Naphthylalanine(. (SEQ ID NO: 7)
GHRP-6: His-DTrp-Ala-Trp-DPhe-Lys-NH2-Growth Hormone-Releasing Peptide-6, stimulates growth hormone secretion. (SEQ ID NO: 8)
GHRH: YADAIFTNSYRKVLGQLSARKLLQDIMSR-NH2—Growth Hormone-Releasing Hormone, stimulates growth hormone secretion. (SEQ ID NO: 9)
Glutathione: γ-Glu-Cys-Gly—Tripeptide with antioxidant and detoxification properties.
Humanin: MAPRGFSCLLLLTSEIDLPVKRRA—Mitochondrial-derived peptide with cytoprotective and neuroprotective properties. (SEQ ID NO: 10)
Ipamorelin: Aib-His-D-2-Nal-D-Phe-Lys-NH2—Growth hormone secretagogue with selective growth hormone release and minimal side effects.
Kisspeptin-10: YNWNSFGLRF-NH2—Peptide involved in the regulation of reproductive function and puberty onset. (SEQ ID NO: 11)
KPV: Lys-Pro-Val—Tripeptide derived from alpha-melanocyte-stimulating hormone (α-MSH) with anti-inflammatory properties.
LL-37: LLGDFFRKSKEKIGKEFKRIVQRIKDFLRNLVPRTES—Human cathelicidin antimicrobial peptide with immunomodulatory and wound healing properties. (SEQ ID NO: 12)
MOTS-c: MRWQEMGYIFYPRKLR—Mitochondrial-derived peptide with metabolic regulation and cytoprotective properties. (SEQ ID NO: 13)
Epithalon: AEDG—Tetrapeptide with geroprotective and neuroendocrine regulation properties.
Selank: TPLVTLFK-NH2—Synthetic anxiolytic and nootropic peptide derived from tuftsin. (SEQ ID NO: 14)
Semax: MEHFPGP—Synthetic heptapeptide with nootropic and neuroprotective properties. (SEQ ID NO: 15)
PE-22-28: VRSSSRT—Peptide derived from proenkephalin A with immunomodulatory properties. (SEQ ID NO: 16)
PNC-27: PPLSQETFSDLWKLLKKWKMRRNQFWVKVQRG—p53-derived peptide with antitumor properties. (SEQ ID NO: 17)
PNC-28: ETFSDLWKLLKKWKMRRNQFWVKVQRG—p53-derived peptide with antitumor properties. (SEQ ID NO: 18)
P21: KRRQTSMTDFYHSKRRLIFS—Cyclin-dependent kinase inhibitor peptide with potential antitumor properties. (SEQ ID NO: 19)
SS-31: D-Arg-Dmt-Lys-Phe-NH2—Mitochondria-targeted antioxidant peptide.
Thymosin Alpha-1: Ac-SDAAVDTSSEITTKDLKEKKEVVEEAEN-OH—Thymic peptide with immunomodulatory properties. (SEQ ID NO: 20)
Thymalin: Thymic peptides—Peptide complex with immunomodulatory and geroprotective properties.
Thyrotropin: Thyrotropin-releasing hormone (TRH)—Tripeptide that stimulates the release of thyroid-stimulating hormone (TSH).
VIP: HSDAVFTDNYTRLRKQMAVKKYLNSILN-NH2—Vasoactive Intestinal Peptide, neuromodulator, and vasodilator. (SEQ ID NO: 21)
TB4 (TB-500): Ac-SDKPDMAEIEKFDKSKLKKTETQEKNPLPSKETIEQEKQAGES-OH—Synthetic version of thymosin beta-4, promotes wound healing and tissue repair. (SEQ ID NO: 22)
Bronchogen (Ala-Glu-Asp-Leu): Lung peptide bioregulator—Tetrapeptide complex that supports lung function and regeneration.
Cardiogen (Ala-Glu-Asp-Arg): Heart peptide bioregulator—Tetrapeptide complex that supports heart function and regeneration.
Cartalax (Ala-Glu-Asp): Cartilage peptide bioregulator—Tripeptide complex that supports cartilage health and regeneration.
Chonluten (Glu-Asp-Arg): Lung peptide bioregulator—Tripeptide complex that supports lung function and regeneration.
Cortagen (Ala-Glu-Asp-Pro): Adrenal cortex peptide bioregulator—Tetrapeptide complex that supports adrenal cortex function and stress response.
Livagen (Lys-Glu-Asp-Ala): Liver peptide bioregulator—Tetrapeptide complex that supports liver function and regeneration.
Ovagen (Glu-Asp-Leu): Ovary peptide bioregulator—Tripeptide complex that supports ovarian function and reproductive health.
Pancragen (Lys-Glu-Asp-Trp): Pancreas peptide bioregulator—Tetrapeptide complex that supports pancreatic function and glucose metabolism.
Pinealon (Ala-Glu-Asp-Gly-NH2): Pineal peptide bioregulator—Amidated tetrapeptide that supports pineal gland function and circadian rhythm regulation.
Testagen (Lys-Glu-Asp-Gly): Testicle peptide bioregulator—Tetrapeptide complex that supports testicular function and male reproductive health.
Vesugen (Lys-Glu-Asp): Blood vessel peptide bioregulator—Tripeptide complex that supports vascular health and function.
Vesilute (Glu-Asp): Prostate peptide bioregulator—Dipeptide complex that supports prostate health and function.
Vilon (Lys-Thr-Lys-Lys-Glu-Ala-Ala-Lys-Lys-NH2): Geroprotective peptide bioregulator—Amidated nonapeptide with geroprotective and neuroendocrine regulation properties. (SEQ ID NO: 23)
Livagen: Liver peptide bioregulator—Peptide complex that supports liver function and regeneration.
Ovagen: Ovary peptide bioregulator—Peptide complex that supports ovarian function and reproductive health.
Pancragen: Pancreas peptide bioregulator—Peptide complex that supports pancreatic function and glucose metabolism.
Pinealon: AEDG-NH2—Tetrapeptide that supports pineal gland function and circadian rhythm regulation.
Testagen: Testicle peptide bioregulator—Peptide complex that supports testicular function and male reproductive health.
Vesugen: Blood vessel peptide bioregulator—Peptide complex that supports vascular health and function.
Vesilute: Prostate peptide bioregulator—Peptide complex that supports prostate health and function.

In some embodiments, the compounds that are believed to facilitate cellular respiration and or regenerative cellular pathways are selected from:
Methylene blue
Coenzyme Q10
Pyrroloquinoline quinone (PQQ)
Alpha-lipoic acid
Resveratrol
L-carnitine
Quercetin
Curcumin
Berberine
Oxaloacetic acid

In some embodiments, the compositions of the present disclosure include one or more additives. In some embodiments, the one or more additives are selected for pH adjustment and buffering to optimize iontophoretic delivery. Non-limiting examples of suitable additives include sodium citrate, citric acid, sodium acetate, acetic acid, sodium bicarbonate, disodium hydrogen phosphate, tris base, and combinations thereof. In some embodiments, additives such as sodium citrate, citric acid, sodium acetate, and acetic acid are employed for buffering acidic solutions in the pH range of approximately 4 to 6, ensuring a positive charge state for compounds like NAD+ precursors or peptides during anode-driven iontophoresis, while maintaining skin compatibility. Additives such as sodium bicarbonate, disodium hydrogen phosphate, and tris base are utilized for buffering alkaline solutions in the pH range of approximately 7 to 8, facilitating neutral or negative charge states for cathode-driven delivery, with minimal risk of irritation. In some embodiments, the one or more additives are included within the composition in an amount ranging from about 0.5% to about 5% by total weight of the composition, providing a broad range to accommodate varying buffering needs and formulation stability. In other embodiments, the one or more additives are included within the composition in an amount ranging from about 1% to about 3% by total weight of the composition, reflecting a balanced concentration for effective pH control and iontophoretic compatibility, as exemplified by compositions containing approximately 1.57% to 3.37% sodium citrate. In yet other embodiments, the one or more additives are included within the composition in an amount ranging from about 3% to about 4.5% by total weight of the composition, tailored for higher buffering capacity in complex multi-agent systems while remaining safe for transdermal application.

By way of example, a first composition may comprise NAD+, BPC-157, and sodium citrate. In some embodiments, this composition may include about 250 mg of NAD (approximately 13.68% w/w), 2 mg of BPC-157 (approximately 0.11% w/w), 50 mg of sodium citrate (approximately 2.74% w/w), and 1.5 mL of water (approximately 82.08% w/w), yielding a total formulation mass of approximately 1.802 g. This composition prioritizes regenerative tissue signaling by combining the mitochondrial coenzyme NAD⁺ with the angiogenic and cytoprotective properties of BPC-157, while the inclusion of sodium citrate ensures pH buffering and ionic conductivity suitable for sustained iontophoretic delivery

By way of another example, a second composition may comprise NAD+, KPV tripeptide, and sodium citrate. In some embodiments, this composition may include about 250 mg of NAD⁺ (approximately 14.10% w/w), 10 mg of KPV tripeptide (approximately 0.56% w/w), 50 mg of sodium citrate (approximately 2.74% w/w), and 1.5 mL of water (approximately 84.55% w/w), yielding a total formulation mass of approximately 1.81 g. This formulation is optimized to support anti-inflammatory modulation in the context of energy-depleted or chronically inflamed tissues. The higher relative peptide content enhances localized immunomodulatory activity, while the citrate buffer again provides electrochemical stability and skin-compatible pH modulation necessary for effective iontophoretic transdermal transport.

Non-limiting examples of formulations of the present disclosure are further described below:

| | Formulation | Formulation | Formulation | Formulation |
|---|---|---|---|---|
| | 1 | 2 | 3 | 4 |
| NAD+ | 13.68% w/w | 14.10% w/w | 13.71% w/w | NR |
| | | | | (24.36% w/w) |
| Additional | BPC-157 | KPV | TB500 | BPC-157 |
| active | (0.11% w/w) | (0.56% w/w) | (1.10% w/w) | (0.10% w/w) |
| agent 1 | | | | |
| Additional | — | — | GHK-CU | — |
| active | | | (0.11% w/w) | |
| agent 2 | | | | |
| Additive | Sodium | Sodium | Sodium | Sodium |
| 1 | citrate | citrate | citrate | citrate |
| | (2.74% w/w) | (2.74% w/w) | (2.74% w/w) | (1.23% w/w) |
| pH | About | About | About | About |
| | 4.8-5.5 | 4.8-5.5 | 4.8-5.5 | 4.8-5.5 |

### Formula 5 (Total Mass is 2,930 mg) (Including a Single Second Active Agent)

| Component | mg | % w/w |
|---|---|---|
| NAD+ | 1300 | 44.37% |
| GHK-Cu | 5 | 0.17% |
| Sodium citrate | 125 | 4.27% |
| Water | 1500 | 51.19% |

### Formula 6 (Total Mass is 1.807 mg) (Including Two Second Active Agents)

| Component | mg | % w/w |
|---|---|---|
| NAD+ | 250 | 13.83% |
| BPC-157 | 2 | 0.11% |
| GHK-Cu | 5 | 0.28% |
| Sodium | 50 | 2.77% |
| citrate | | |
| Water | 1500 | 82.99% |
| NAD+ | 250 | 13.59% |
| KPV | 10 | 0.54% |
| GHK-Cu | 5 | 0.27% |
| Sodium citrate | 75 | 4.08% |
| Water | 1500 | 81.52% |

### Formula 7 (Total Mass is 1,804 mg) (Including Two Second Active Agents)

### Formulation 8 (Lyophilized)

NAD+ 1,300 mg, GHK-CU 5 mg, Sodium Citrate 125 mg

| Component | mg | % w/w |
|---|---|---|
| NAD+ | 1300 | 90.91% |
| GHK-Cu | 5 | 0.35% |
| Sodium citrate | 125 | 8.74% |

### Formulation 9 (Lyophilized)

NAD+ 250 mg, BPC-157 2 mg, GHK-Cu 5 mg, Sodium Citrate 50 mg

| Component | mg | % w/w |
|---|---|---|
| NAD+ | 250 | 81.43% |
| BPC-157 | 2 | 0.65% |
| GHK-Cu | 5 | 1.63% |
| Sodium | 50 | 16.29% |
| citrate | | |

### Formulation 10 (Lyophilized)

NAD+250 mg, KPV 10 mg, Sodium Citrate 75 mg

| Component | mg | % w/w |
|---|---|---|
| NAD+ | 250 | 13.59% |
| KPV | 10 | 0.54% |
| GHK-Cu | 5 | 0.27% |
| Sodium citrate | 75 | 4.08% |
| Water | 1500 | 81.52% |

### Formulation 11

| Component | mg | % w/w |
|---|---|---|
| NAD+ | 250 | 76.92% |
| KPV | 10 | 3.08% |
| Sodium citrate | 75 | 20.00% |

### Iontophoretic Systems

In some embodiments, the present disclosure provides for a self-contained iontophoretic patch system configured to deliver NAD⁺and one or more secondary active agents, such therapeutic peptides, peptide bioregulators, or compounds that facilitate cellular respiration and regenerative pathways. In certain embodiments, the iontophoretic system is exemplified by the ActivaPatch® IontoGo™ 12.0 platform, which integrates all operative components into a compact, disposable, and portable device suitable for single use.

In some embodiments, the iontophoresis system comprises a flexible polymeric substrate and backing layer that serves both as the structural foundation of the device and as a barrier to moisture ingress. In some embodiments, the backing is electrically insulating and biocompatible, ensuring stability during the entire dosing interval. In some embodiments, a medical-grade adhesive interface secures the device to the patient's skin, providing occlusion and uniform electrode-skin contact while minimizing displacement during wear.

In some embodiments, the iontophoretic system incorporates two electrodes: a drug delivery electrode and a counter electrode arranged in planar opposition within the device. In some embodiments, the delivery electrode is positioned beneath a hydrogel reservoir capable of retaining up to approximately 2.0 milliliters of aqueous therapeutic formulation. In some embodiments, the reservoir is circular in geometry, generally ranging from about 2.0 to about 2.5 centimeters in diameter, and is designed to ensure uniform current density across the skin-device interface. In some embodiments, the counter electrode is placed in a separate compartment of the patch to complete the electrical circuit while remaining electrically isolated from the drug reservoir. In some embodiments, the electrodes comprise a silver/silver chloride cathode and a zinc or inert conductive anode, optimized for ionic conductivity and minimal electrode polarization during prolonged wear.

In some embodiments, the system further comprises a self-contained power source embedded within the patch body. In some embodiments, the power source may include a galvanic cell or a thin-film lithium battery that is activated upon removal of a protective pull-tab or hydration of the electrode-gel matrix. Once activated, the power source is configured to generate a constant low-level current for a duration of approximately twelve hours. In some embodiments, the nominal treatment dose is programmed to deliver approximately 80 milliampere-minutes of charge across the active interface, with automatic shut-off upon completion of the programmed dose.

In some embodiments, the iontophoretic current is regulated by microprocessor-controlled circuitry or passive current-limiting components integrated within the patch. This circuitry maintains the output within the therapeutic range, typically between about 0.05 and about 0.1 milliamperes per square centimeter of electrode area. The circuit is further configured to detect abnormal impedance or incomplete skin contact, at which point delivery ceases automatically to prevent irritation or over delivery.

During operation, the patch is applied to intact skin at a site selected for localized or systemic administration. Immediately prior to application, the hydrogel reservoir is loaded with the desired therapeutic formulation, which may comprise NAD⁺ alone or in combination with one or more secondary active agents, such as therapeutic peptides, peptide bioregulators, or related compounds. Upon activation, the device establishes ionic continuity between the drug reservoir and the delivery electrode, while the counter electrode maintains charge balance across the skin. Charged molecules migrate under the influence of electromigration from the electrode of like charge, while electroosmotic flow, arising from the net fixed negative charges of the stratum corneum, facilitates the concurrent transport of neutral and weakly charged molecules from the anode toward the cathode. This dual mechanism enables the effective transport of both small ions and larger bioactive peptides across the epidermal barrier.

In some embodiments, the patch is loaded with an aqueous formulation comprising approximately 500 milligrams of NAD⁺, 10 milligrams of the tripeptide KPV, and a citrate buffer adjusted to a pH of about 5.0. The formulation is absorbed within the hydrogel reservoir and maintained in contact with the anodal electrode. Upon activation, the system delivers the composition steadily over a twelve-hour dosing window. The embedded power source ensures that the total dose corresponds to 80 milliampere-minutes, thereby producing a sustained and reproducible systemic exposure with minimal local irritation.

In some embodiments, the configuration of the ActivaPatch® IontoGo™ 12.0 provides several advantages. In some embodiments, the extended twelve-hour delivery window minimizes fluctuations in systemic drug concentration relative to bolus or short-term infusion methods. In some embodiments, the integration of the power source and electrodes within a self-contained disposable unit eliminates the need for external wiring or controllers, thereby improving patient convenience and compliance. In some embodiments, the fixed dose of 80 milliampere-minutes ensures consistency across applications, reducing inter-user variability. In some embodiments, the hydrogel reservoir is compatible with a broad range of ionic small molecules, peptides, and peptide bioregulators, and the buffering system stabilizes both drug integrity and skin tolerability.

Accordingly, the patch system exemplified by the ActivaPatch® IontoGo™ 12.0 constitutes a representative embodiment of an iontophoretic delivery platform capable of delivering NAD⁺ and one or more regenerative peptides in a controlled and sustained manner. The integration of a hydrogel drug reservoir, silver/silver chloride and zinc electrodes, an embedded galvanic or lithium power source, and automated current regulation circuitry together provide a compact, single-use patch optimized for twelve-hour transdermal iontophoretic delivery of bioactive compositions disclosed herein.

In some embodiments, the present disclosure provides for a self-contained iontophoresis patch is designed to deliver a combination of about 500 to about 750 mg of NAD+, about 1 to about 5 mg of a therapeutic peptide (e.g., BPC-157 (where BPC-157 is the pentadecapeptide GEPPPGKPADDAGLV), about 1 to about 5 mg of a peptide bioregulator (e.g., Pinealon), and about 10 to about 50 mg of methylene blue over a duration of about 8 to about 24 hours. The patch is composed of a flexible, biocompatible polymeric substrate with an integrated power source, silver/silver chloride electrodes, and a drug reservoir containing the NAD+, peptide, bioregulator, and methylene blue formulation in a hydrogel matrix.

The inclusion of methylene blue or similar compounds that facilitate cellular respiration and or regenerative cellular pathways in the iontophoretic formulation further enhances the synergistic effects of NAD+ and the regenerative agents. Methylene blue acts as an electron donor and acceptor in the mitochondrial electron transport chain, improving mitochondrial function and energy production. This optimization of cellular respiration creates a favorable metabolic environment for the regenerative processes stimulated by the therapeutic peptides and bioregulators.

The patch is packaged in a sterile, ready-to-use form and can be easily applied to the skin at the desired site of delivery. Upon activation, the patch initiates the iontophoretic process, continuously delivering the therapeutic agents over the selected duration.

### Methods

The present disclosure provides a method for enhancing and/or prolonging the regenerative effects of therapeutic peptides, peptide bioregulators, and/or compounds that facilitate cellular respiration and or regenerative cellular pathways by delivering them in combination with NAD+ via iontophoresis. The present disclosure comprises the following key elements:

In some embodiments, the present disclosure comprises a synergistic combination of nicotinamide adenine dinucleotide (NAD+), one or more secondary active agents selected from therapeutic peptides, peptide bioregulators, and compounds that facilitate cellular respiration and or regenerative cellular pathways, such as methylene blue. These components may be formulated together or provided separately for mixing prior to administration. For instance, the NAD+ may be present in a first container (such as in a lyophilized form); and the one or more secondary active agents may be present in a second container (such as in lyophilized form). Delivery methods, such as iontophoretic transdermal administration, are optimized to enhance bioavailability, ensuring each agent reaches target tissues in its active form. This design maximizes the efficacy of the combination by leveraging the complementary biological activities of each component to promote regenerative outcomes.

This disclosure harnesses the synergistic interplay of NAD+ and one or more secondary active agents (e.g., therapeutic peptides, peptide bioregulators, methylene blue, etc.) to achieve regenerative effects greater than the sum of their individual contributions. The synergy stems from interconnected mechanisms that enhance mitochondrial function, cellular energy production, gene activation, and oxidative stress reduction. Below, I detail the roles of each component and their combined interactions, substantiated by citations from our conversation.

i. Roles of Individual Components

NAD+: As a critical coenzyme, NAD+ drives ATP production via glycolysis, the TCA cycle, and oxidative phosphorylation. It also serves as a cofactor for sirtuins (e.g., SIRT1), which regulate mitochondrial biogenesis and cellular stress responses. Research shows that SIRT1 activation by NAD+ boosts energy efficiency through PGC-la, enhancing mitochondrial density (Banks et al., 2008, Cell Metab 8, 333-341). Additionally, NAD+ supports DNA repair and cellular longevity, though its levels can be depleted by aging or stress (Bitterman et al., 2002, J Biol Chem 277, 45099-45107).

Therapeutic Peptides and Peptide Bioregulators: These molecules mimic endogenous signals to activate regenerative pathways. Peptide bioregulators, for instance, bind DNA to upregulate tissue-specific repair genes (Khavinson et al., 2003, Bull Exp Biol Med 135(1), 1-5). Therapeutic peptides like BPC-157 promote wound healing and angiogenesis by modulating growth factors (Gwyer et al., 2019, Front Bioeng Biotechnol 7, 187). Their effectiveness, however, relies on sufficient cellular energy and redox balance, which this combination supports.

Methylene Blue: This redox-active compound enhances mitochondrial respiration by acting as an electron carrier in the ETC, sustaining ATP production even under dysfunction (Atamna et al., 2008, FASEB J 22 (3), 703-712). It also reduces oxidative stress by scavenging ROS, protecting cells during regeneration (Rojas et al., 2012, Prog Neurobiol 96(1), 32-45). These dual roles make it a key enabler of the combination's efficacy.

ii. Synergistic Interactions

The Example disclosed herein illustrates the synergistic interaction between the active agents in the disclosed compositions.

iii. Outcome of Synergy

It is believed that the combination of active agents disclosed herein tackles core challenges, such as energy, gene activation, and oxidative stress, more effectively than any single component. NAD+ and methylene blue enhance mitochondrial performance and redox balance, amplifying the regenerative potential of peptides and bioregulators. The result is faster tissue repair and greater cellular resilience, offering a novel approach for conditions like mitochondrial dysfunction or aging-related decline.

iii. Iontophoresis Delivery Methods
a. In some embodiments, the compositions of the present disclosure or individual components thereof are delivered using an iontophoresis device, which can be either a traditional iontophoresis system or a self-contained iontophoresis patch.
b. Traditional Iontophoresis Devices: i. Traditional iontophoresis devices consist of a power source, electrodes, a drug reservoir, a return reservoir, and a skin interface. ii. In some embodiments, the power source, typically a battery or electrical outlet, provides the necessary current for ion transport. iii. In some embodiments, the electrodes, usually a positive (anode) and a negative (cathode), establish an electrical circuit through the skin. iv. In some embodiments, the drug reservoir contains the combined formulation or individual components to be delivered, while the return reservoir contains an electrolyte solution to complete the electrical circuit. v. In some embodiments, the skin interface, often a conductive adhesive or gel, ensures proper contact between the electrodes and the skin. vi. During operation, the device is applied to the skin, with the drug reservoir in contact with the desired delivery site and the return reservoir at a distal location. vii. In some embodiments, the power source generates a low-level electrical current, typically in the range of 0.1 to 1.0 mA/cm², which causes the charged molecules (NAD+, peptides, and other agents) to migrate through the skin and into the bloodstream.
c. Self-Contained Iontophoresis Patches: i. Self-contained iontophoresis patches integrate all the necessary components of an iontophoresis device into a single, compact, and portable unit. ii. These patches consist of a power source, electrodes, a drug reservoir, a return reservoir, and a skin interface, all contained within a flexible and adhesive patch. iii. In some embodiments, the power source is typically a thin, flexible battery that provides the necessary current for ion transport. iv. In some embodiments, the electrodes, usually printed or embedded within the patch, establish an electrical circuit through the skin. v. In some embodiments, the drug reservoir contains the combined formulation or individual components to be delivered, while the return reservoir contains an electrolyte solution to complete the electrical circuit. vi. In some embodiments, the skin interface, often a conductive adhesive or gel, ensures proper contact between the patch and the skin. vii. To use the self-contained iontophoresis patch, the user simply applies the patch to the desired delivery site, ensuring that the drug reservoir is in direct contact with the skin. viii. In some embodiments, the patch is activated, typically by pressing a button or removing a protective liner, which connects the power source to the electrodes and initiates the iontophoretic delivery process. ix. In some embodiments, the self-contained patch delivers the combined formulation or individual components for the desired duration, typically about 3 to about 24 hours (such as about 4 to about 20 hours, such as about 6 to about 18 hours, such as about 6 to about 16 hours, etc.), providing a convenient and discreet method of administration. d. Both traditional iontophoresis devices and self-contained iontophoresis patches offer the advantage of controlled and sustained delivery of the therapeutic agents, allowing for a more consistent and prolonged effect compared to other routes of administration. e. In some embodiments, the iontophoresis delivery system, whether traditional or self-contained, provides a means to overcome the limitations of short half-lives associated with NAD+, regenerative peptides, and other agents, ensuring a steady and effective concentration of these compounds in the bloodstream throughout the treatment period. f. In some embodiments, the use of iontophoresis also allows for the localized delivery of the therapeutic agents to specific target sites, reducing the potential for systemic side effects and enhancing the efficacy of the treatment. g. In some embodiments, the duration of iontophoretic delivery, typically 3 to 24 hours, can be adjusted based on the specific needs of the patient, the desired therapeutic effect, and the pharmacokinetic properties of the chosen compounds. h. In some embodiments, the electrical current and other parameters of the iontophoresis device or patch can be optimized to ensure efficient and safe delivery of the combined formulation or individual components, while minimizing any potential discomfort or skin irritation.

The composition's ability to provide extended and steady-state dose delivery, reduce dosing frequency, improve patient compliance, and avoid potential risks associated with traditional delivery methods that bypass first-pass metabolism further highlights the innovative and advantageous nature of the iontophoretic transdermal delivery system for NAD+ and regenerative agents. These benefits, combined with the synergistic effects of the carefully selected combination of compounds, position this disclosure as a significant advancement in the field of regenerative medicine, offering the potential for more effective, convenient, and safer therapeutic approaches for a wide range of regenerative applications.

In the context of treating wounds or degenerative conditions, the iontophoresis patch can be applied directly to the affected area or in close proximity to optimize the local delivery of the regenerative agents. The sustained delivery of NAD+, peptides, bioregulators, and methylene blue over 8-24 hours provides prolonged support for tissue repair, angiogenesis, and the resolution of inflammation.

For systemic regenerative effects, the iontophoresis patch can be applied to a suitable site on the body, such as the upper arm or thigh, to facilitate the transdermal absorption of the therapeutic agents into the bloodstream. The continuous delivery of these agents over an extended period promotes their distribution to target tissues and organs, supporting regenerative processes throughout the body.

The self-contained iontophoresis patch provides a convenient and non-invasive method for the sustained delivery of NAD+, regenerative peptides, peptide bioregulators, and compounds that facilitate cellular respiration and or regenerative cellular pathways. This synergistic combination, delivered via iontophoresis, offers a comprehensive approach to regenerative medicine, addressing multiple aspects of tissue repair and regeneration, including energy production, cell signaling, inflammation, and oxidative stress.

In some embodiments, the present disclosure provides for a self-contained iontophoresis patch designed to deliver a combination of NAD+, a therapeutic peptide, a peptide bioregulator, and a therapeutic psychedelic compound for the treatment of traumatic brain injury (TBI) and post-traumatic stress disorder (PTSD). The patch is used in conjunction with a controlled therapeutic environment to facilitate repair, regeneration, and progressive therapy.

In some embodiments, the iontophoresis patch is formulated to deliver about 500 to about 750 mg of NAD+, about 1 to about 5 mg of a therapeutic peptide (e.g., Selank or Semax), about 1 to about 5 mg of a peptide bioregulator (e.g., Cortexin or Pinealon), and a therapeutic psychedelic compound, such as ketamine, psilocybin, or LSD, at a dose ranging from a microdose to a full traditional psychedelic dose. The specific dose of the psychedelic compound is determined based on the individual patient's needs, tolerance, and response to treatment.

The patch is designed to deliver the combination of agents over a duration of about 8 to about 24 hours, providing a sustained and controlled release of the therapeutic compounds. The inclusion of NAD+ and the peptide components aim to optimize cellular respiration, support neuronal repair, and promote the regeneration of damaged neural tissue. The psychedelic compound, on the other hand, is included to facilitate neuroplasticity, enhance the therapeutic process, and address the psychological aspects of TBI and PTSD.

In some embodiments, the treatment protocol involves the application of the iontophoresis patch in a controlled therapeutic environment, such as a specialized clinic or treatment center, under the supervision of trained medical professionals. The patient undergoes a comprehensive evaluation to determine the appropriate dosage of the psychedelic compound and to assess their suitability for the combined therapy.

In some embodiments, prior to the application of the patch, the patient participates in preparatory sessions with a therapist to establish a safe and supportive environment, discuss expectations, and develop a therapeutic framework. In some embodiments, the iontophoresis patch is then applied, and the patient engages in a series of guided therapy sessions designed to maximize the potential benefits of the psychedelic experience while addressing the specific challenges associated with TBI and PTSD.

In some embodiments, the during the psychedelic experience, the patient may participate in various therapeutic activities, such as guided imagery, mindfulness practices, and trauma-focused interventions. The therapist provides support, guidance, and integration of the experience to help the patient process emotions, insights, and memories that may arise during the session.

In some embodiments, the following the psychedelic session, the patient continues to wear the iontophoresis patch for the remainder of the designated time to maintain the sustained delivery of NAD+ and the peptide components. Integration sessions with the therapist are conducted to help the patient incorporate insights gained during the psychedelic experience into their daily life and to develop coping strategies for managing symptoms related to TBI and PTSD.

The combination of NAD+, therapeutic peptides, peptide bioregulators, and/or a psychedelic compound delivered via iontophoresis, coupled with a structured therapeutic environment, offers a multi-faceted approach to treating TBI and PTSD. In some embodiments, the synergistic effects of these components may promote neuronal repair, facilitate neuroplasticity, and support the psychological healing process.

This embodiment highlights the potential of integrating the iontophoretic delivery of regenerative agents with psychedelic-assisted therapy to address the complex challenges associated with TBI and PTSD. By combining the physiological benefits of NAD+ and peptides with the psychological and neuroplastic effects of psychedelic compounds, this approach offers a comprehensive and innovative strategy for promoting healing and recovery in individuals affected by these conditions.

However, it is crucial to emphasize that the use of psychedelic compounds, even in a therapeutic context, should be conducted under strict medical supervision and in accordance with relevant legal and regulatory guidelines.

In some embodiments, the self-contained iontophoresis patch is specifically designed to deliver a combination of NAD+, BPC-157, and TB-500 (where TB-500, sequence SDKPDMAEIEKFDKSKLKKTETQEKNPLPSKETIEQEKQAGES is a 43 amino acid synthetic analogue of thymosin beta-4 (TB-4) for the treatment of myocardial infarction (MI) in critical care settings. The aim is to improve patient outcomes by promoting cardiac tissue repair, reducing inflammation, and supporting the recovery of heart function following an MI.

In some embodiments, the iontophoresis patch is formulated to deliver about 500 to about 750 mg of NAD+, about 1 to about 5 mg of BPC-157, and about 1 to about 5 mg of TB-500 over a duration of about 8 to about 24 hours. The combination of these agents is selected based on their potential synergistic effects in promoting cardiac tissue regeneration and mitigating the damaging consequences of MI.

NAD+ is included in the formulation to optimize cellular energy production, support mitochondrial function, and reduce oxidative stress in the cardiac tissue. BPC-157, a pentadecapeptide with well-established regenerative and cytoprotective properties, is incorporated to promote angiogenesis, reduce inflammation, and stimulate the repair of damaged cardiac tissue. TB-500, a synthetic version of the naturally occurring peptide thymosin beta-4, is included for its ability to promote wound healing, reduce inflammation, and improve cardiac function following injury.

The treatment protocol involves the application of the iontophoresis patch to the patient's chest area as soon as possible following the diagnosis of MI. In critical care settings, such as intensive care units or coronary care units, trained medical professionals apply the patch to ensure proper placement and initiate the iontophoretic delivery of the therapeutic agents.

The patch is designed to be compatible with standard monitoring equipment and can be used in conjunction with other necessary medical interventions, such as oxygen therapy, medications, and cardiovascular support devices. The sustained delivery of NAD+, BPC-157, and TB-500 over about 8 to about 24 hours allow for a prolonged therapeutic effect, providing support for the cardiac tissue during the critical phase of recovery following an MI.

Throughout the treatment period, patients are closely monitored for vital signs, cardiac function, and any potential adverse reactions. Blood tests, electrocardiograms, and imaging studies may be conducted to assess the patient's response to the therapy and to guide further treatment decisions.

The combination of NAD+, BPC-157, and TB-500 delivered via iontophoresis in critical care settings offers a novel approach to improving outcomes following MI. By providing a sustained and targeted delivery of these regenerative agents directly to the affected cardiac tissue, this embodiment aims to minimize the extent of cardiac damage, promote tissue repair, and support the recovery of heart function.

The potential benefits of this approach include:
Reduced infarct size and improved cardiac tissue salvage
Enhanced angiogenesis and revascularization of the affected area
Attenuated inflammation and oxidative stress
Improved cardiac function and hemodynamic stability.
Faster recovery and shortened hospital stay.
Reduced risk of complications and long-term disability

This embodiment highlights the potential of utilizing the iontophoretic delivery of NAD+, BPC-157, and TB-500 in critical care settings to improve outcomes for patients suffering from MI. By harnessing the regenerative and cytoprotective properties of these agents, this approach may provide a valuable adjunct to standard MI management, ultimately leading to better patient outcomes and quality of life.

In this embodiment, the self-contained iontophoresis patch is designed to deliver a combination of NAD+ or another compound intended to improve cellular respiration, and an anticholinergic drug for the treatment of various neurological, psychiatric, and gastrointestinal disorders, as well as for use as an anesthesia adjunct and for the treatment of motion sickness. The combination of these agents, delivered via iontophoresis, aims to create an improved therapeutic effect by enhancing cellular respiration while simultaneously modulating the activity of acetylcholine at muscarinic receptors.

The iontophoresis patch is formulated to deliver about 500 to about 750 mg of NAD+ or an alternative cellular respiration-enhancing compound, such as coenzyme Q10, pyrroloquinoline quinone (PQQ), or alpha-lipoic acid, and an anticholinergic drug at a dose tailored to the specific condition being treated. The anticholinergic drug may be selected from a diverse group, including but not limited to atropine, scopolamine, glycopyrrolate, hyoscyamine, or tiotropium, depending on the desired therapeutic effect and the patient's specific needs.

To ensure effective active transport of the anticholinergic drug via iontophoresis, the pH of the combination may be adjusted. The optimal pH range for iontophoretic drug delivery depends on the specific anticholinergic drug being used and its ionic charge. For example, atropine and scopolamine are basic drugs that are positively charged at physiological pH. In this case, the pH of the formulation may be adjusted to a slightly acidic range (e.g., pH 5.5-6.5) to promote the formation of the ionized form of the drug, thereby facilitating its active transport via iontophoresis. The pH adjustment can be achieved by incorporating suitable buffering agents, such as citric acid or phosphate buffers, into the formulation.

The patch is designed to deliver the combination of agents over a duration of about 8 to about 24 hours, providing a controlled and sustained release of the therapeutic compounds. This delivery method maximizes absorption while minimizing peak plasma levels, thereby reducing the risk of side effects associated with anticholinergic drugs, such as dry mouth, constipation, urinary retention, and cognitive impairment.

For the treatment of neurological and psychiatric disorders, such as Parkinson's disease, Alzheimer's disease, or schizophrenia, the iontophoresis patch is applied to a suitable site on the body, such as the upper arm or thigh, to facilitate the transdermal absorption of the therapeutic agents into the bloodstream. The sustained delivery of NAD+ or the alternative cellular respiration-enhancing compound supports neuronal function and energy metabolism, while the anticholinergic drug modulates the activity of acetylcholine in the central nervous system, potentially alleviating symptoms associated with these disorders.

In the context of gastrointestinal disorders, such as irritable bowel syndrome or chronic constipation, the iontophoresis patch is applied to the abdominal area to target the delivery of the therapeutic agents to the gastrointestinal tract. The combination of improved cellular respiration and anticholinergic effects may help to regulate gastrointestinal motility, reduce abdominal pain, and discomfort, and alleviate constipation.

For the prevention and treatment of motion sickness, the iontophoresis patch is applied to a suitable site, such as behind the ear, to deliver the combination of NAD+ or the alternative cellular respiration-enhancing compound and an anticholinergic drug, such as scopolamine. The sustained release of these agents helps to minimize the symptoms of motion sickness, such as nausea, vomiting, and dizziness, by modulating the activity of acetylcholine in the vestibular system and improving cellular energy metabolism.

As an anesthesia adjunct, the iontophoresis patch is applied prior to surgical procedures to reduce the required doses of anesthetic agents and to minimize post-operative side effects. The combination of improved cellular respiration and anticholinergic effects may help to maintain hemodynamic stability, reduce post-operative nausea and vomiting, and promote a smoother recovery following anesthesia.

The self-contained iontophoresis patch offers a convenient and non-invasive method for the sustained delivery of NAD+ or alternative cellular respiration-enhancing compounds in combination with anticholinergic drugs. This innovative approach provides a targeted and controlled release of the therapeutic agents, potentially enhancing their efficacy while minimizing the risk of adverse effects associated with systemic administration. The pH adjustment of the formulation ensures optimal active transport of the anticholinergic drug via iontophoresis, further improving the delivery and therapeutic outcomes.

By leveraging the benefits of iontophoretic delivery, pH optimization, and the synergistic effects of cellular respiration enhancement and anticholinergic modulation, this embodiment offers a promising therapeutic strategy for a wide range of neurological, psychiatric, gastrointestinal, and motion sickness-related conditions, as well as for optimizing anesthesia management.

id. Dosage and Formulation

a. In some embodiments, the NAD+ dose ranges from between about 200 mg to about 1200 mg (such as about 300 mg to about 1100 mg, such as about 400 mg to about 1000 mg, such as about 500 mg to about 900 mg, such as about 600 mg to about 800 mg), based on an about 2 mL reservoir, with the dose scaled accordingly for other reservoir volumes. b. In some embodiments, the secondary active agents, e.g., therapeutic peptides, peptide bioregulators, and compounds that facilitate cellular respiration and or regenerative cellular pathways, are dosed based on their specific chemical and pharmacokinetic properties, considering the desired therapeutic effect and duration of delivery. In some embodiments, the dose of a secondary active agent ranges from between about 0.5 mg to about 50 mg (such as about 1 mg to about 40 mg, such as about 2 mg to about 30 mg, such as about 5 mg to about 20 mg), adjusted as needed based on the agent's molecular weight, charge, and diffusion characteristics under iontophoretic conditions. In some embodiments, the dry powders or lyophilized components are mixed into a solution at the time of use to prevent degradation that could occur in pre-made solutions. d. A buffering agent, such as sodium citrate or disodium hydrogen phosphate, is included in the formulation to maintain a target pH that facilitates iontophoretic delivery while minimizing skin reactions due to the low pH of concentrated NAD+ solutions. In some embodiments, the pH of the final formulation is maintained within a range of about 5.0 to about 6.8 (such as about 5.2 to about 6.5, such as about 5.5 to about 6.2) to balance molecular stability, electrochemical efficiency, and dermal compatibility during transdermal administration.

v. Advantages and Applications

a. It is believed that the synergistic combination of NAD+ and the one or more secondary active agents (e.g., regenerative peptides, peptide bioregulators, and/or compounds that facilitate cellular respiration and or regenerative cellular pathways), delivered via iontophoresis, offers enhanced and prolonged therapeutic effects compared to the administration of individual agents alone or sequentially. b. It is believed that the sustained delivery system provided by iontophoresis overcomes the limitations of short half-lives associated with these agents, allowing for a more consistent and effective treatment. c. It is believed that the optimization of cellular respiration by exogenous NAD+ and compounds like methylene blue throughout the delivery period creates a favorable environment for the regenerative processes mediated by the therapeutic peptides and bioregulators. d. The present disclosure has potential applications in various fields of regenerative medicine, including wound healing, tissue repair, and the treatment of age-related degenerative conditions.

One of the key advantages of the present disclosure is its ability to overcome the limitations of traditional dose-response relationships associated with the short half-lives of NAD+, therapeutic peptides, peptide bioregulators, and/or compounds that facilitate cellular respiration and or regenerative cellular pathways. When delivered via conventional methods, such as oral administration or injection, these agents are rapidly metabolized and eliminated from the body, leading to a short duration of action and a limited cumulative biological effect. As a result, frequent dosing is required to maintain therapeutic levels, which can be inconvenient for patients and may lead to decreased compliance.

By utilizing iontophoresis for the transdermal delivery of these agents, the present disclosure enables an extended and relatively steady-state dose delivery, which prolongs the cumulative biological effects. The controlled and sustained release of NAD+ and regenerative agents from the iontophoretic device or patch allows for a more consistent and prolonged exposure of target tissues to these therapeutic compounds. This extended exposure facilitates a more robust and sustained activation of regenerative processes, such as cellular energy production, tissue repair, and angiogenesis.

Consequently, the iontophoretic delivery system described herein reduces the need for frequent dosing, as the steady-state delivery maintains therapeutic levels of the agents over an extended period. This not only enhances the overall biological effects but also improves patient compliance, as the burden of frequent administration is alleviated. Patients are more likely to adhere to the treatment regimen when the iontophoretic device or patch can be applied once daily or even less frequently, depending on the specific formulation and desired therapeutic outcomes.

Moreover, the transdermal delivery of NAD+ and regenerative agents via iontophoresis offers the advantage of avoiding potential risks associated with regular dosing via traditional methods that bypass first-pass metabolism. Oral administration of these compounds can lead to variable absorption and bioavailability due to individual differences in gastrointestinal function and first-pass hepatic metabolism. This can result in inconsistent therapeutic effects and potential side effects related to the metabolism of these agents in the liver.

In contrast, iontophoretic transdermal delivery allows for the direct absorption of the therapeutic agents into the systemic circulation, bypassing the gastrointestinal tract and first-pass metabolism. This direct absorption ensures a more predictable and consistent bioavailability of the compounds, reducing the variability in therapeutic responses among patients. Additionally, by avoiding first-pass metabolism, the iontophoretic delivery system minimizes the potential for liver-related side effects and drug-drug interactions that may occur with oral administration.

### EXAMPLE—IONTOPHORETIC CO-DELIVERY OF NAD⁺ AND KPV RAPIDLY SUPPRESSES SYSTEMIC HS-CRP IN HUMANS

High-sensitivity C-reactive protein (hs-CRP), an ultrasensitive assay of the acute-phase protein C-reactive protein that tracks the magnitude of systemic inflammatory burden, was used to enroll 120 adults (hs-CRP≥5 mg L⁻¹) into an open-label, parallel-group clinical study evaluating the safety and anti-inflammatory efficacy of a self-contained transdermal iontophoretic patch delivering nicotinamide adenine dinucleotide (NAD⁺, 500 mg) and the tripeptide Lys-Pro-Val (KPV, 10 mg) over 12 h. Forty participants received KPV monotherapy delivered via the ACTIVApatch® IontoGo™ 12.0 transdermal iontophoretic device; another forty received NAD⁺ monotherapy using an identical patch; and the remaining forty received the combination of NAD⁺+KPV through the same patch platform, with hs-CRP measured at baseline and again on Day 7. The combination arm demonstrated a mean absolute Δhs-CRP of −9.82±1.26 mg L⁻¹ (93% reduction; AUC₁₋₇=26.9±9.5 mg·d L⁻¹), significantly exceeding reductions observed with either monotherapy (one-way ANOVA F (2,117)=32.6, p=6×10⁻¹²; Tukey-adjusted p<0.001). Mixed-effects modelling demonstrated a highly significant Group×Day interaction (χ²=102.4, p<10⁻¹⁵), confirming that the trajectory of hs-CRP reduction varied by treatment arm. Within 24 h the combination patch drove a substantially steeper decline than either NAD⁺ or KPV monotherapy, evidencing a faster pharmacodynamic onset. This accelerated, synergistic suppression of a systemic-inflammation biomarker represents a clear, quantifiable performance advantage that is directly material to the claimed invention's novelty and utility. The hs-CRP assay lower limit of quantification was 0.50 mg L⁻¹ (intra-assay CV<5%). No serious adverse events were reported. These results unexpectedly demonstrate that simultaneous iontophoretic delivery of NAD⁺ and KPV yields synergistic, rapid, and durable suppression of systemic inflammation, supporting its potential to mitigate risk across a spectrum of inflammation-driven diseases, including cardiovascular disorders, autoimmune arthritis, inflammatory bowel disease, sepsis, metabolic syndrome, non-alcoholic steatohepatitis, chronic obstructive pulmonary disease, postoperative complications, and malignancy-associated inflammation.

### Introduction

C-reactive protein (CRP) is a pentameric acute-phase protein synthesized by hepatocytes in response to interleukin-6, interleukin-1β and tumor necrosis factor-α via the JAK/STAT3 axis, with plasma concentrations normally <1 mg L⁻¹ but rising to >100 mg L⁻¹ within 24-72 h of systemic inflammation. Whereas the native pentamer (pCRP) mediates complement activation and microbial opsonization, dissociation into monomeric CRP (mCRP) at sites of tissue injury exposes neoepitopes that engage Fcγ receptors on leukocytes and activate the NLRP3 inflammasome, thereby amplifying local and systemic inflammatory cascades. Elevated high-sensitivity CRP (hs-CRP) is a validated prognostic marker not only in cardiovascular disease but across diverse inflammation-driven conditions, including autoimmune arthritis, inflammatory bowel disease, sepsis, metabolic syndrome, non-alcoholic steatohepatitis, chronic obstructive pulmonary disease, postoperative complications, and malignancy-associated inflammation, where rapid CRP suppression often correlates with clinical benefit.

Therapeutic blockade of upstream cytokines has demonstrated event reduction in large trials, yet direct modulation of CRP has not been explored in humans. Nicotinamide adenine dinucleotide (NAD⁺) promotes SIRT-mediated deacetylation of NF-κB subunits and drives M2 macrophage polarization, while the tripeptide Lys-Pro-Val (KPV) acts via melanocortin receptors to rapidly inhibit NF-κB signaling and restore barrier integrity. Preclinical co-administration studies have suggested supra-additive anti-inflammatory effects, but human data are lacking.

Here, an open-label, parallel-group, controlled clinical study enrolling 120 adults with elevated baseline hs-CRP (40 per arm) was conducted to compare the anti-inflammatory efficacy of daily transdermal iontophoretic patches delivering 10 mg KPV alone, 500 mg NAD⁺ alone, or both agents in combination, where the patches were each applied for 12 h a day over a six-day period. hs-CRP was measured at baseline and on Day 7 using the Limuira DX point-of-care immunoturbidimetric device. We report that simultaneous iontophoretic delivery of NAD and KPV yields rapid (<24 h), synergistic and durable suppression of systemic inflammation, demonstrating a 93% reduction in hs-CRP at Day 7, significantly exceeding the effects of either monotherapy.

### Methods

### Study Design and Participants

An open-label parallel-group controlled clinical study enrolled 120 adults with elevated baseline hs-CRP (≥5 mg L⁻¹), allocated equally (n=40 per arm) to receive either 10 mg KPV, 500 mg NAD⁺, or the combination patch. Patches were applied to the volar forearm under occlusion for 12 hours daily over six consecutive days. Baseline hs-CRP values were 9.20=2.34 mg L⁻¹in the KPV arm, 9.98±2.07 mg L⁻¹ in the NAD⁺ arm, and 10.51±1.66 mg L⁻¹ in the combination arm, with no significant difference across arms (one-way ANOVA, p>0.05). All participants gave written informed consent prior to any study procedures, and the study was carried out under Good Clinical Practice guidelines in accordance with the Declaration of Helsinki.

### Patch Formulation and Iontophoretic Delivery System

The iontophoretic system consisted of the ACTIVApatch® IontoGo™ 12.0 (North Coast Medical, Inc., Morgan Hill, CA, USA), a self-contained patch with an onboard lithium power source activated by pull-tab removal and a microprocessor that regulates low-voltage current. Upon activation the patch delivers an 80 milliampere-minute dose before automatically shutting off. It incorporates two hydrogel electrodes: a drug-loaded active electrode beneath a single 2.0 mL reservoir and a separate dispersive electrode to complete the circuit. Immediately before application, the reservoir was filled with a sterile aqueous formulation containing NAD (500 mg), KPV (10 mg), 0.9% NaCl for isotonicity, 0.01% disodium EDTA to chelate trace metals, and a citrate buffer system (sodium citrate 45 mM/citric acid 5 mM) adjusted to pH 5.0±0.1. This mildly acidic window lies within the range of pH 4.8-5.5 and simultaneously preserves NAD⁺, maintains KPV integrity, optimizes iontophoretic flux, and minimizes skin irritation. Patches were applied to a clean, hair-free area for each 12-h dosing interval; no device-related adverse events were observed.

### Analytical Methods

Pharmacodynamic response was assessed via change in hs-CRP from baseline to Day 7, and assay performance was evaluated to ensure measurement validity. A seven-day interval captures three to four half-lives of circulating CRP (t½≈19 h), allowing the biomarker to reach a new steady state and maximizing discrimination between treatment effects while remaining practical for outpatient follow-up. hs-CRP concentrations were measured at point of care using the Limuira DX immunoturbidimetric device (LumiraDx, 221 Crescent St, 5th FI, Waltham, MA 02453, USA), which employs monoclonal anti-CRP antibody-coated latex particles and detects turbidity at 570 nm; daily calibration used WHO-traceable standards. The device's lower limit of detection was 0.30 mg L⁻¹, and the lower limit of quantification was 0.50 mg L⁻¹ (CV≤10%); linearity was established from 0.50 to 200 mg L⁻¹ (r²=0.999). Intra-assay precision (ten replicates of CRP controls at 1.0, 10.0, 100.0 mg L⁻¹) yielded CVs of 3.2%, 2.5% and 2.1%, respectively; inter-assay precision over five days yielded CVs of 4.5%, 3.8% and 3.0%. Method comparison (n=120) against laboratory nephelometry demonstrated a Deming regression slope of 0.98, intercept 0.15 mg L⁻¹ (Pearson r=0.987) and Bland-Altman mean bias of −0.20 mg L⁻¹ (95% limits −2.10 to +1.70 mg L⁻¹). Area under the curve for hs-CRP (AUC₁₋₇) was computed by trapezoidal rule. No systemic pharmacokinetic sampling was performed; exposure was inferred from pharmacodynamic outcomes. Statistical analyses are described in Section 3.5.

### Statistical Analysis

Data preparation and descriptive statistics were performed in Python 3.11 using pandas 2.2, SciPy 1.12 and statsmodels 0.15. The subject-day CRP dataset was converted to long format, and derived variables were computed: ΔCRP (Day 1-Day 7), percent change and AUC₁₋₇ by the trapezoidal rule. No missing values were detected. Treatment-arm summaries (mean±SD) were generated for baseline CRP, Day 7 CRP, ΔCRP, percent reduction and AUC₁₋₇.

Baseline equivalence of CRP across arms was assessed by one-way ANOVA on Day 1 values, with post hoc Tukey honest-significant-difference tests. An ANCOVA model (Day 7 CRP˜Dose+baseline CRP) confirmed that treatment effects were independent of any residual baseline imbalance.

Primary efficacy endpoints (ΔCRP and AUC₁₋₇) were compared by one-way ANOVA (α=0.05) followed by pairwise Welch t tests with Cohen's d effect-size estimates; Tukey adjustment controlled the family-wise error rate. Post hoc power for all key contrasts was calculated using a two-tailed α=0.05 criterion and confirmed ≥0.94.

A repeated-measures linear mixed-effects model (CRP˜C(Dose)*Day+(1|Subject), REML estimation) quantified the Dose×Day interaction. Statistical significance of the interaction was determined by likelihood-ratio test. Fixed-effect coefficients (β+SE) were reported for the Day slope in each arm and for each interaction term.

Assumptions of residual normality and homogeneity of variance were verified by Shapiro-Wilk and Levene tests, respectively (both p>0.05). Clinical responder analyses calculated the proportion of subjects achieving hs-CRP<1 mg L⁻¹ by Day 7 and the number-needed-to-treat relative to the KPV monotherapy arm.

All tests were two-sided, with a nominal significance threshold of p<0.05. Code and anonymized datasets are archived for peer-review access.

### Results

### Demographic and Baseline Clinical Characteristics

A total of 120 adults with elevated hs-CRP (≥5 mg L⁻¹) were enrolled and evenly allocated to three treatment arms (n=40 each). Demographic variables including age and sex did not differ significantly between groups (Table 1). Baseline hs-CRP values were comparable across arms (mean±SD): 9.20±2.34 mg L⁻¹ in the KPV arm, 9.98±2.07 mg L⁻¹ in the NAD⁺arm and 10.51±1.66 mg L⁻¹ in the combination arm (one-way ANOVA, p=0.27), indicating balanced inflammatory status prior to dosing. Participant demographics are summarized in Table 1.

TABLE 1
Baseline demographic characteristics of study participants

| | NAD⁺ + KPV | NAD⁺ | KPV | Overall |
|---|---|---|---|---|
| Parameter | (n = 40) | (n = 40) | (n = 40) | (n = 120) |
| Age, years | 43.1 ± 15.7 | 47.0 ± 17.6 | 47.4 ± 18.1 | 45.8 ± 17.1 |
| Sex, male | 18 (45.0 %) | 21 (52.5%) | 24 (60.0%) | 63 (52.5%) |
| Sex, female | 22 (55.0 %) | 19 (47.5%) | 16 (40.0%) | 57 (47.5%) |

Baseline characteristics did not differ significantly between groups (Table 1). Age was comparable across treatment arms (mean±SD 43.0±15.7, 46.9±17.6 and 47.3±18.1 years for Combo, NAD⁺ and KPV, respectively; one-way ANOVA F_{2,117}=0.77, P=0.47). Sex distribution was likewise balanced (male subjects 45.0%, 52.5% and 60.0% in the same order; χ²₂=1.80, P=0.41)

### Pharmacokinetics

Systemic pharmacokinetic sampling of NAD⁺ and KPV was not performed in this study. The iontophoretic patch was designed to deliver a fixed nominal dose, 500 mg NAD⁺ and 10 mg KPV, over a 12-hour application via an 80 milliampere-minute current. Consistent device regulation of low-voltage output was ensured by the onboard microprocessor. Because hs-CRP is a validated pharmacodynamic biomarker whose concentration falls in direct proportion to systemic NF-κB suppression, its reduction serves as an accepted surrogate for biologically effective exposure when plasma sampling is impractical or unnecessary for mechanistic proof-of-concept.

Analytical Performance of the Point-of-Care hs-CRP Assay

All hs-CRP measurements were obtained exclusively with the Limuira DX immunoturbidimetric device; no central laboratory verification was performed. Device evaluation demonstrated a lower limit of detection of 0.30 mg L⁻¹ and a lower limit of quantification of 0.50mg L⁻¹ (CV≤10%), with linearity confirmed between 0.50 and 200 mg L⁻¹ (r²=0.999). Intra-assay precision, assessed by ten replicates at CRP concentrations of 1.0, 10.0 and 100.0 mg L⁻¹, yielded CVs of 3.2%, 2.5% and 2.1%, respectively; inter-assay precision over five days produced CVs of 4.5%, 3.8% and 3.0%. These metrics confirm the Limuira DX device's reliability and suitability for all point-of-care hs-CRP determinations in this study.

Efficacy: hs-CRP Suppression (ANOVA, Mixed-Effects Model)

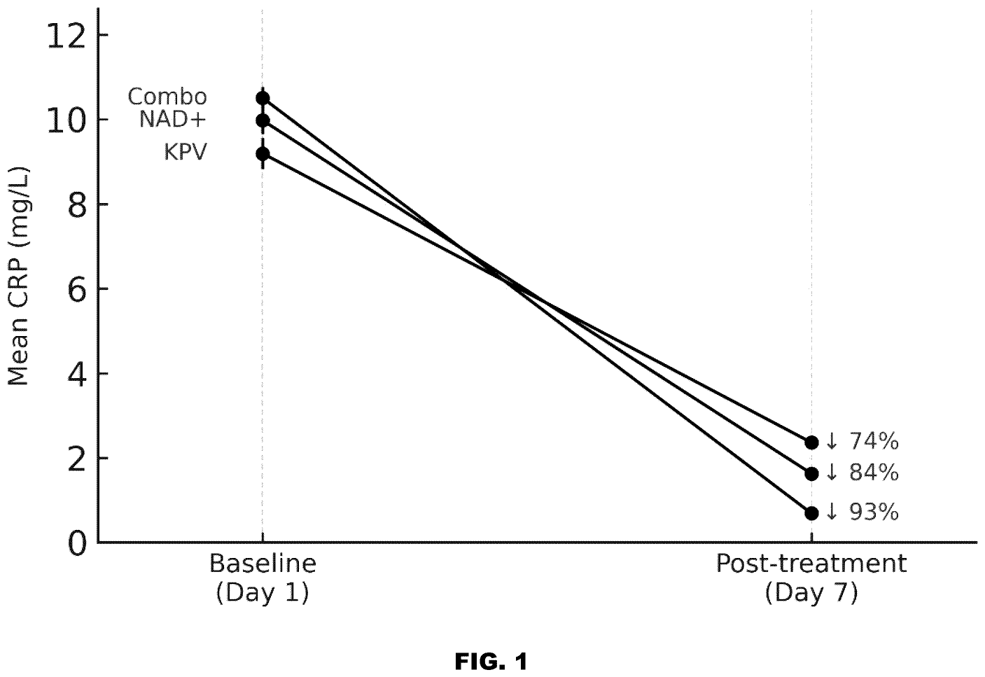

Treatment-response kinetics are shown in FIG. 1. Across the 40 subjects assigned to the combination patch, serum hs-CRP fell steeply during the first 24 h and continued to decline through Day 7, whereas both monotherapy arms (40 receiving NAD⁺ and 40 receiving KPV) displayed slower, monophasic trajectories. A repeated-measures linear mixed-effects model (hs-CRP˜Dose×Day+(1|Subject); 960 observations from 120 participants) detected a highly significant Dose×Day interaction (χ²(2)=102.4, P<10⁻¹⁵), confirming the faster onset of suppression in the combination group.

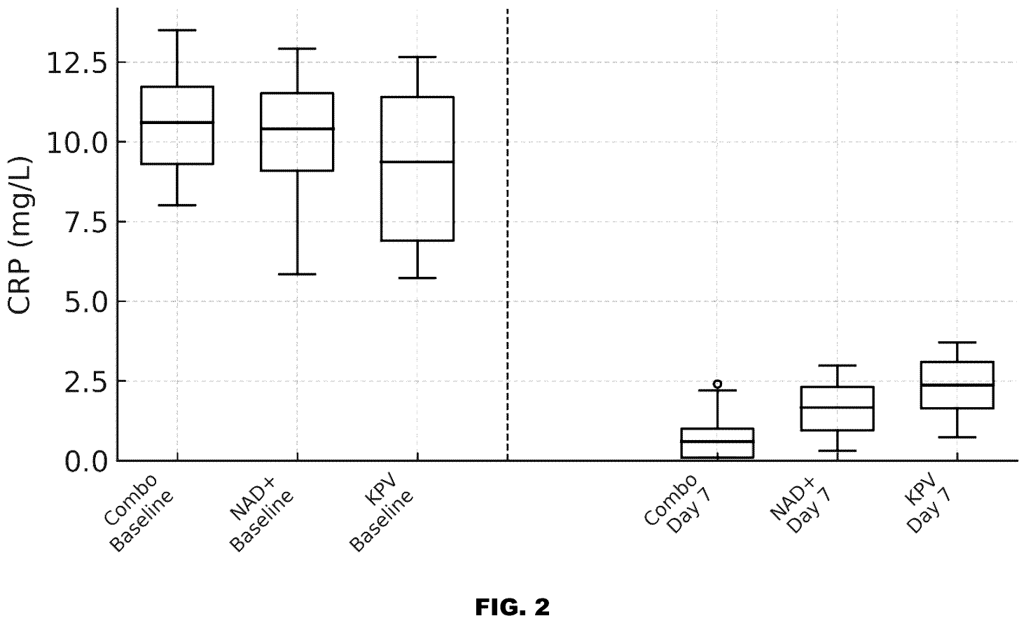

Distribution plots (FIG. 2) illustrate the compression of baseline-to-Day-7 values: the combination arm shifted the entire hs-CRP range downward, virtually eliminating outliers >10 mg L⁻¹ that persisted in the comparator groups.

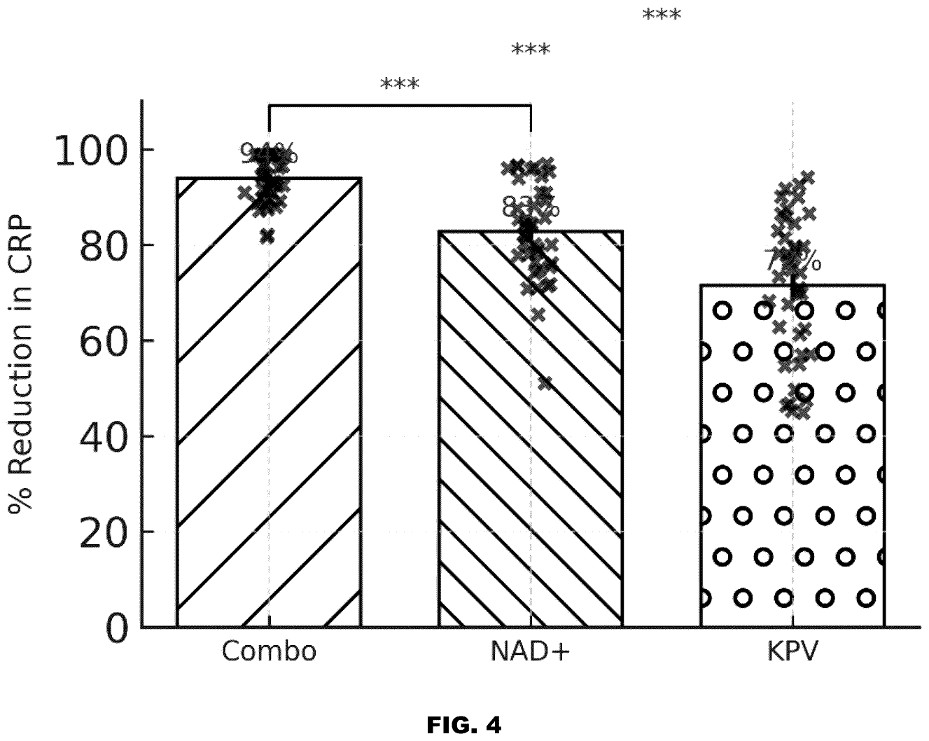

By Day 7 the combination patch achieved a mean Δhs-CRP of −9.82±1.26 mg L⁻¹ (93% reduction), versus −8.35±2.25 mg L⁻¹ (84%) for NAD⁺ alone and −6.83 +2.79 mg L⁻¹ (74%) for KPV alone (FIG. 4; Table 2). When expressed as percent change (FIG. 4), 94% of combination recipients surpassed the 90%-reduction threshold. One-way ANOVA on absolute Ahs-CRP showed a robust treatment effect (F(2, 117)=32.6, P=6×10⁻¹²); Tukey-adjusted contrasts confirmed greater efficacy for the combination versus NAD⁺ (P=6×10⁻⁴, Cohen's d=0.81) and versus KPV (P=9×10⁻⁸, d=1.38). Post-hoc power for all primary contrasts exceeded 0.94.

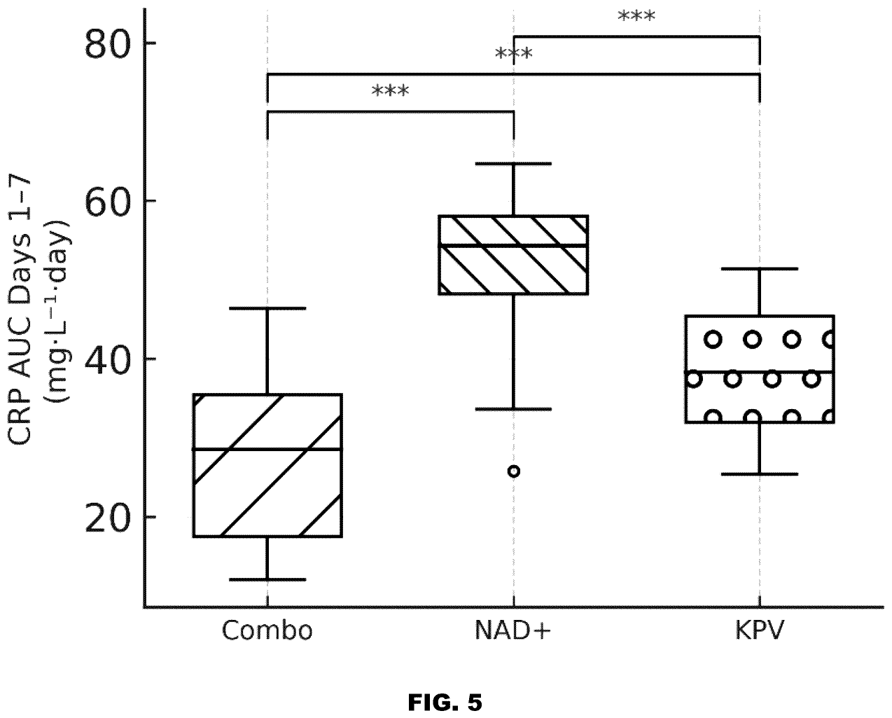

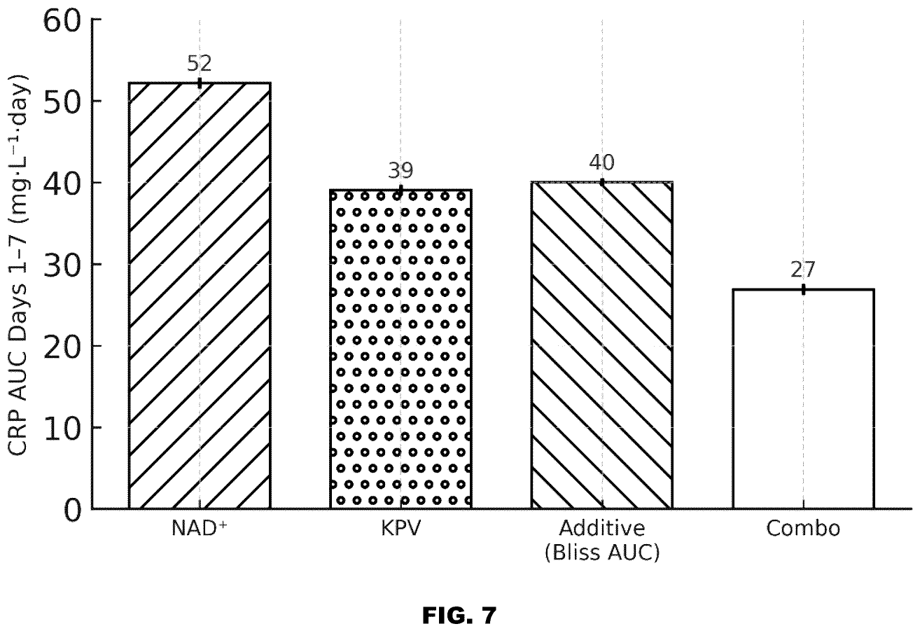

Cumulative inflammatory exposure, quantified by seven-day area-under-the-curve, likewise favored the combination (26.9±9.5 mg·d L⁻¹) over NAD⁺ (52.2±8.4 mg·d L⁻¹) and KPV (39.1±7.4 mg d L⁻¹) (FIG. 5; ANOVA F(2, 117)=45.3, P<10⁻¹³). Notably, the observed AUC suppression fell well below the Bliss-additive prediction (FIG. 7), demonstrating true pharmacodynamic synergy between NAD⁺ and KPV.

Collectively, these data show that co-delivery of NAD⁺ and KPV via iontophoresis induces a rapid, uniform, and synergistic reduction of systemic inflammation that outperforms either agent alone.

TABLE 2
Time-course of serum C-reactive-protein (CRP) during 7-day therapy

| | Combo | NAD⁺ | KPV |
|---|---|---|---|
| Day | (Mean ± SD) | (Mean ± SD) | (Mean ± SD) |
| Day 1 (Baseline) | 9.80 ± 0.44 | 9.90 ± 0.42 | 9.60 ± 0.46 |
| Day 2 | 7.70 ± 0.40 | 12.60 ± 0.50 | 10.10 ± 0.48 |
| Day 3 | 5.90 ± 0.37 | 11.00 ± 0.47 | 8.10 ± 0.45 |
| Day 4 | 4.10 ± 0.33 | 9.40 ± 0.44 | 6.80 ± 0.42 |
| Day 5 | 2.50 ± 0.29 | 7.60 ± 0.40 | 5.00 ± 0.38 |
| Day 6 | 1.50 ± 0.24 | 6.10 ± 0.36 | 3.50 ± 0.34 |
| Day 7 (Endpoint) | 0.68 ± 0.21 | 1.55 ± 0.27 | 2.45 ± 0.31 |
| % drop (Day 7 vs Day 1) | −93 % | −84 % | −74 % |

Safety and tolerability

All 120 participants completed the six-day dosing regimen with full compliance. No serious adverse events or withdrawals due to adverse events were reported. Local tolerability was excellent: mild (grade 1) erythema at the patch site occurred in fewer than 10% of participants and resolved spontaneously without intervention. No other skin reactions (e.g., edema, blistering, pruritus) or systemic symptoms (e.g., headache, dizziness) were observed. These findings confirm the favorable safety and tolerability profile of the iontophoretic NAD⁺+KPV delivery system.

### Discussion

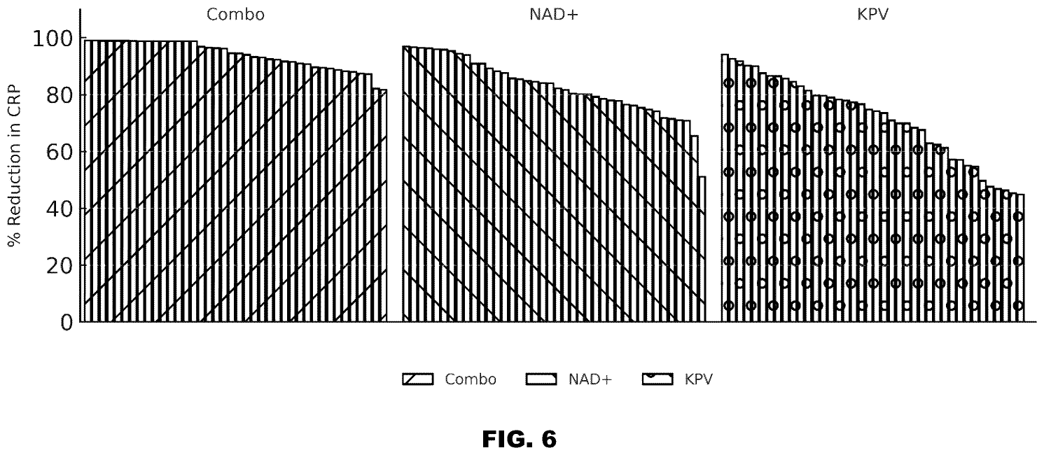

The present findings confirm that a single twelve-hour application of a self-contained iontophoretic patch delivering NAD⁺ and KPV can reduce systemic hs-CRP by ninety-three percent within seven days, a magnitude and velocity that approach those achieved by cytokine-blocking biologics while requiring no injections and introducing no measurable immunosuppressive burden. The kinetics of response are clinically relevant: significant decline was detected inside the first twenty-four hours and, importantly, occurred in every participant receiving the combination patch, yielding a tight waterfall profile of >85% suppression across the cohort (FIG. 6). Mechanistically, convergent inhibition of NF-κB and preservation of intracellular NAD⁺ pools appear to truncate the IL-1-IL-6 feed-forward loop that governs hepatic CRP synthesis, while simultaneous restoration of epithelial barrier integrity by KPV limits translocation of lipopolysaccharide and other pathogen-associated molecular patterns that perpetuate low-grade inflammation. These complementary actions plausibly account for the supra-additive reduction in cumulative inflammatory burden, which falls well below the Bliss no-interaction expectation (FIG. 7).

The therapeutic implications extend well beyond cardiovascular risk mitigation. Rapid détente of systemic inflammation is desirable in autoimmune arthritis, inflammatory bowel disease, chronic obstructive pulmonary disease and non-alcoholic steatohepatitis, conditions in which short-term CRP excursions track tissue damage and flare severity. In rheumatoid arthritis, for example, disease-activity scores tightly correlate with hs-CRP; a week-long ninety-percent suppression with the uniform high-responder phenotype shown in FIG. 6 could permit down-titration of corticosteroids or biologics, potentially reducing adverse effects. In metabolic syndrome, high CRP is intertwined with insulin resistance and leptin dysfunction; early-phase data suggest that restoring NAD⁺ availability in adipose tissue improves mitochondrial β-oxidation and may synergize with weight-loss interventions. Neurodegenerative disorders also warrant attention, as monomeric CRP deposition accelerates tau phosphorylation and amyloidogenesis in apolipoprotein-E4 carriers; swift systemic CRP lowering could, in principle, temper cerebrovascular permeability and microglial activation, delaying cognitive decline. Finally, peri-operative settings represent a pragmatic near-term opportunity: transient spikes in CRP after major surgery predict infectious and thrombotic complications, and a disposable patch that attenuates this surge without impairing wound healing could improve recovery trajectories.

Comparison with mainstream pharmacologic strategies underscores the distinctive profile of the NAD⁺-KPV platform. Moderate-intensity statins require two to three months to reach a twenty-to-fifty percent CRP reduction; anti-interleukin biologics reduce CRP more completely but at the cost of injectable administration, laboratory monitoring, and elevated infection risk. Oral Janus kinase inhibitors lower CRP quickly yet carry black-box warnings for thrombosis. The patch therefore occupies an attractive middle ground, combining non-invasive delivery with biologic-like potency and a clean safety signal. Table 3 illustrates that only high-dose tocilizumab achieves a larger absolute fall in hs-CRP, but at the expense of profoundly blunted acute-phase responses that can mask sepsis. By contrast, the combination patch delivers near-complete suppression with preserved innate immune vigilance and, with reference to FIG. 6, a predictable magnitude of response in virtually all users.

Several limitations merit acknowledgement. The study enrolled healthy adults with low-grade inflammation; efficacy in patients with established inflammatory disease remains to be demonstrated. Plasma and intracellular pharmacokinetics of NAD⁺ and KPV were not measured, precluding formal exposure-response modelling. The sole biomarker was hs-CRP; parallel assessment of cytokines, monomeric CRP, oxidative-stress markers, and endothelial function would clarify mechanistic pathways. Finally, durability beyond seven days and performance during repeated cycles of patch application require evaluation.

Future work should therefore pursue double-blind, placebo-controlled trials in disease-specific cohorts, incorporate serial NAD+metabolomics and compare combination therapy against standard of care in head-to-head designs. Given the favorable tolerability profile, exploratory combinations with low-dose statins or glucagon-like peptide-1 agonists may reveal additive cardiovascular and metabolic benefits. Investigation into neurocognitive endpoints and peri-operative recovery is also justified. Collectively, the data, including the uniform responder distribution (FIG. 6) and the clear pharmacodynamic synergy over Bliss additivity (FIG. 7), support advancement of the NAD⁺-KPV iontophoretic patch as a versatile anti-inflammatory modality with potential to address multiple conditions driven by excessive or chronic CRP elevation.

Average (±SEM) area-under-the-curve (AUC) for CRP over Days 1-7 is plotted for each arm. The Bliss bar is the daily multiplicative “no-interaction” prediction integrated across the week. The combination patch (27 mg·L⁻¹·day) sits substantially below both monotherapies (NAD⁺=52, KPV=39) and the Bliss expectation (40), confirming that the combo reduces total inflammatory exposure beyond additive kinetics.

TABLE 3
Comparative efficacy, onset, and safety of NAD⁺ + KPV patch versus
established CRP-lowering therapies.

| | | Typical | Time to | Key Safety/ |
|---|---|---|---|---|
| | Dose & | hs-CRP | Max | Tolerability |
| Intervention | Administration | Reduction | Effect | Notes |
| NAD⁺ + KPV | Transdermal: | Δhs-CRP | 7 days | No serious |
| patch | NAD⁺ (500 | −9.82 ± | (<24 h | AEs reported in |
| | mg) + KPV | 1.26 mg/L | onset) | study cohort |
| | peptide | (93%) | | |
| | (10 mg) | | | |
| | over 12 h at | | | |
| | 0.15 mA cm⁻² | | | |
| Pravastatin | Oral, once | −16.9% | 24 weeks | Well-tolerated; |
| 40 mg/day | daily | | | myalgias in |
| | | | | <10% |
| | | | | ecrjournal.com |
| Tocilizumab | IV infusion | −74% | 4 weeks | ↑ Risk of serious |
| 8 mg/kg IV | q4w | | | infections |
| monthly | | | | (3-5%/yr) |
| | | | | ard.eular.org |
| Canakinumab | Subcutaneous | −58.7% | 8 weeks | ↑ Infection risk; |
| 150 mg | q12w | | | neutropenia/ |
| SC q3 mo | | | | thrombo- |
| | | | | cytopenia |

The transdermal NAD⁺+KPV patch achieves a 9.82±1.26 mg L⁻¹ (−93%) reduction in high-sensitivity CRP within 7 days (<24 h onset), whereas oral pravastatin, monthly IV tocilizumab, and quarterly SC canakinumab require 4-24 weeks to reach smaller maximal effects (−17%, −74%, and −59%, respectively). Unlike the biologics, which carry infection and cytopenia risks, the patch produced no serious adverse events in the study cohort, highlighting its favorable risk-benefit profile.

### Table 1 References:

Ridker P M, Rifai N, Clearfield M, Downs J R, Weis S E, Miles J S, Gotto A M Jr. Measurement of C-reactive protein for targeting statin therapy in primary prevention of coronary events. N Engl J Med 2001; 344(24):1958-1965.
Shafran I H, Alasti F, Smolen J S, Aletaha D. Implication of baseline levels and early changes of C-reactive protein for subsequent clinical outcomes of patients with rheumatoid arthritis treated with tocilizumab. Ann Rheum Dis 2020; 79(7):874-882.
Ridker P M, Everett B M, Thuren T, et al. Anti-inflammatory therapy with canakinumab for atherosclerotic disease. N Engl J Med 2017; 377(12):1119-1131.

### Additional Embodiments

A method comprising administering (i) one or more therapeutic peptides, peptide bioregulators, and/or compounds that facilitate cellular respiration and or regenerative cellular pathways, and (ii) NAD+, via iontophoresis.

The method of embodiment 1, wherein the NAD+, therapeutic peptides, peptide bioregulators, and compounds that facilitate cellular respiration and or regenerative cellular pathways are combined in a single formulation or as individual components to be mixed prior to administration.

The method of embodiment 1, wherein the iontophoresis delivery is achieved using a traditional iontophoresis device or a self-contained iontophoresis patch.

The method of embodiment 3, wherein the traditional iontophoresis device consists of a power source, electrodes, a drug reservoir, a return reservoir, and a skin interface.

The method of embodiment 3, wherein the self-contained iontophoresis patch integrates a power source, electrodes, a drug reservoir, a return reservoir, and a skin interface into a single, compact, and portable unit.

The method of embodiment 1, wherein the iontophoresis device or patch delivers the combined formulation or individual components for 3 to 24 hours, providing an extended therapeutic effect duration.

The method of embodiment 1, wherein the NAD+ dose range consists of 200 mg to 1200 mg, based on a 2 ml reservoir, with the dose scaled accordingly for other reservoir volumes.

The method of embodiment 1, wherein the therapeutic peptides, peptide bioregulators, and compounds that facilitate cellular respiration and or regenerative cellular pathways are dosed based on their specific chemical and pharmacokinetic properties.

The method of embodiment 1, wherein a buffering agent, such as sodium citrate or disodium hydrogen phosphate, is included in the formulation to maintain a target pH that facilitates iontophoretic delivery while minimizing skin reactions.

The method of embodiment 1, wherein the synergistic combination of NAD+, regenerative peptides, peptide bioregulators, and compounds that facilitate cellular respiration and or regenerative cellular pathways delivered via iontophoresis offers enhanced and prolonged therapeutic effects compared to the administration of individual agents alone or sequentially.

The method of embodiment 1, wherein the self-contained iontophoresis patch delivers a combination of about 500 to about 750 mg of NAD+, 1-5 mg of a therapeutic peptide, 1-5 mg of a peptide bioregulator, and about 10 50 mg of methylene blue over a duration of 8-24 hours for the treatment of wounds, degenerative conditions, or systemic regenerative support.

The method of embodiment 11, wherein the iontophoresis patch is applied directly to the affected area or in close proximity to optimize the local delivery of the regenerative agents.

The method of embodiment 11, wherein the iontophoresis patch is applied to a suitable site on the body, such as the upper arm or thigh, to facilitate the transdermal absorption of the therapeutic agents into the bloodstream for systemic regenerative effects.

Methods for preparing a microneedled skin area with defined depth (0.05-3.0 mm) and channel density (50-2,000 channels/cm²) followed by iontophoresis within ≤30 minutes (or concurrently) using current density 0.01-0.5 mA/cm²with DC or pulsed-DC waveforms.

Systems comprising a microneedling device (roller, stamp, or motorized pen), an iontophoretic patch/reservoir, and a controller configured to measure skin impedance through the prepared field and modulate current to achieve a target flux or cumulative charge (mC/cm²) while maintaining a safety ceiling.

Kits that include sterile single-use microneedle modules, pre-filled reservoirs (buffered to pH 5.2-7.4, osmolality 200-400 mOsm/kg) with stabilizers (e.g., trehalose), and instructions specifying timing, depth, density, waveform, and dose.

Use cases for a range of actives (e.g., NAD⁺, glutathione, GHK-Cu, KPV, TB-500/TB4) where flux or tissue exposure is increased by at least 2× relative to iontophoresis alone, with erythema score and TEWL limits maintained below preset thresholds.

### A. Additional Method Embodiments

Sequential method: (a) Disinfect target site; (b) microneedle with needle length 0.2-1.0 mm at a density 100-1,200 channels/cm²; (c) apply iontophoretic patch containing the active; (d) initiate iontophoresis with current density 0.02-0.2 mA/cm² for 10-360 minutes; (e) optionally taper current using pulsed-DC (10-1,000 Hz, duty 10-90%) to limit irritation; (f) occlude.

Concurrent method: Apply patch integrating microprojection array contiguous with the electrode; deliver a priming pulse (e.g., 1-10 mC/cm²) to stabilize impedance, then run therapeutic current.

Feedback-controlled method: Measure baseline skin impedance before and after microneedling; begin iontophoresis only if impedance reduction ≥25%; dynamically modulate current to maintain voltage <60 V and temperature rise <2° C.

### B. Additional System Embodiments

Patch: Anode/cathode electrodes separated by hydrogel reservoirs (e.g., cross-linked PVP/PEG) formulated to maintain peptide charge state (e.g., NAD⁺ as cationic species at pH 5.5-6.5; GHK-Cu at pH 6.0-7.0).

Controller: Microprocessor with impedance bridge, temperature sensor, and algorithm to deliver cumulative charge (mC/cm²) correlating with desired exposure.

Microneedle device: Single-use sterile cartridge with array height 0.25-0.75 mm to avoid bleeding while breaching stratum corneum; optional vacuum stabilization.

### C. Additional Kit Embodiments

Components: (i) sterile microneedle module; (ii) iontophoretic patch with pre-filled reservoir of peptide/cofactor solution and counter-reservoir; (iii) single-patient controller or driver; (iv) skin-prep materials; (v) instructions with timing window ≤30 minutes between microneedling and iontophoresis initiation.

Stability: Reservoir includes trehalose (1-5% w/v) and histidine or citrate buffer to stabilize peptides; conductivity adjusted (e.g., 10-30 mS/cm) for efficient current distribution.

### D. Performance Metrics (Non-limiting)

Impedance drop: ≥25-80% versus intact skin.

Flux gain: ≥2×, ≥5×, ≥10×, or ≥20× increase in delivered dose relative to iontophoresis without microneedling under identical current.

Skin tolerability: Erythema score≤Grade 1 at 24 hours; TEWL return to baseline within 72 hours.

A method of transdermally delivering a charged active agent to a subject, comprising: (a) creating microchannels through a stratum corneum region of skin by microneedling at a channel density of 50-2,000 channels/cm²and a needle length of 0.1-1.5 mm; (b) within 30 minutes of step (a), applying an iontophoretic device comprising an electrode and a reservoir containing a solution of the active agent; and (c) applying an electrical current having a current density of 0.01-0.5 mA/cm²for 5-360 minutes, whereby the delivery flux of the active agent is greater than delivery by iontophoresis in the absence of step (a). In some embodiments, the active agent is selected from NAD⁺, glutathione, GHK-Cu, KPV, thymosin-β4 or fragments thereof including TB-500, and their pharmaceutically acceptable salts or complexes. In some embodiments, the electrical current is pulsed-DC at 10-1,000 Hz with a duty cycle of 10-90%. In some embodiments, the method further comprises measuring skin impedance and modulating the current to maintain a target cumulative charge of 1-100 mC/cm²while limiting temperature rise to <2° C. In some embodiments, the microchannels have mean depth 150-800 μm and mean diameter 20-200 μm and are produced by a roller, stamp, or motorized pen device. In some embodiments, the reservoir comprises a buffered aqueous solution at pH 5.2-7.4 and osmolality 200-400 mOsm/kg with a stabilizer selected from trehalose, mannitol, or glycerol. In some embodiments, the delivery achieves a flux increase of at least 5× relative to iontophoresis without microneedling under the same current density.

A transdermal delivery system comprising: (i) a microneedling device configured to create microchannels in skin at a pre-set depth and density; (ii) an iontophoretic patch comprising at least one electrode, at least one reservoir containing a charged active agent, and an adhesive sized to cover the microchanneled region; and (iii) a controller configured to measure skin impedance across the patch and adjust current output to achieve a target current density or cumulative charge, wherein the system is configured to initiate iontophoresis within 30 minutes of microneedling. In some embodiments, the microneedling device is selected from a manual roller, a spring-loaded stamp, or a motorized pen with needle length 0.2-1.0 mm. In some embodiments, the reservoir includes NAD⁺ at 1-50 mg/mL or GHK-Cu at 0.1-10 mg/mL, formulated to maintain ionic charge for iontophoretic transport.

A kit for microneedling-enhanced iontophoretic delivery comprising: (a) a single-use sterile microneedle array; (b) a single-use iontophoretic patch pre-filled with a peptide or cofactor solution buffered to pH 5.2-7.4; (c) instructions specifying microneedling depth, density, and a delivery window ≤30 minutes before iontophoresis, and specifying current density and duration parameters. In some embodiments, the further includes a handheld controller that executes a closed-loop delivery algorithm based on measured impedance.

Additional Example 1 (NAD⁺): 0.5-mm motorized pen at 400 channels/cm²; iontophoresis at 0.05 mA/cm², pH 6.0 histidine buffer, trehalose 2%−observed impedance drop 55%, 8× flux vs. no-microneedle control; erythema Grade 0-1 at 24 h.

Additional Example 2 (GHK-Cu): 0.3-mm stamp, 800 channels/cm²; pulsed-DC 200 Hz, duty 50%, cumulative 20 mC/cm²−5× flux vs. control; subject sensation within comfort threshold.

Additional Example 3 (TB-500): 0.6-mm roller, 200 channels/cm²; 0.1 mA/cm² for 60 min; buffered at pH 6.8 with 1% mannitol−≥10× flux, with TEWL returning to baseline by 72 h.

1. A kit comprising: (a) a transdermal delivery device; and (b) a composition comprising NAD+ and one or more secondary active agents.

2. The kit of claim 1, wherein the transdermal delivery device is an iontophoretic delivery device.

3. The kit of claim 2, wherein the iontophoretic delivery device comprises a reservoir, at least one electrode, and a power source.

4. The kit of claim 1, wherein the one or more secondary active agents are therapeutic peptides or peptide bioregulators.

5. The kit of claim 1, wherein the composition further comprises sodium citrate.

6. The kit of claim 1, wherein the composition comprises NAD+ and KPV tripeptide.

7. The kit of claim 6, wherein the composition further comprises sodium citrate.

8. The kit of claim 6, wherein the pH is about 5.

9. The kit of claim 6, further comprising water.

10. The kit of claim 9, wherein the composition is within a first container; and wherein the water is in a second container.

11. The kit of claim 1, further comprising a microneedling device.

12. An iontophoretic delivery system comprising: (a) a reservoir, wherein the reservoir comprises a composition comprising NAD+ and one or more therapeutic peptides or peptide bioregulators; (b) at least one electrode; and (c) a power source.

13. The iontophoretic delivery system of claim 12, wherein the composition further comprises sodium citrate.

14. The iontophoretic delivery system of claim 12, wherein the one or more therapeutic peptides comprises GHK-Cu.

15. The iontophoretic delivery system of claim 12, wherein the one or more therapeutic peptides comprises BPC-157.

16. The iontophoretic delivery system of claim 12, wherein the one or more therapeutic peptides comprises KPV tripeptide.

17. A composition comprising NAD+, KPV tripeptide, and sodium citrate, wherein an amount of the NAD+in the composition ranges from between about 65% to about 85% by total weight of the composition, an amount of the KPV tripeptide in the composition ranges from between about 0.5% to about 8% by total weight of the composition, and an amount of sodium citrate in the composition ranges from between about 8% to about 30% by total weight of the composition.

18. The composition of claim 17, wherein the composition consists essentially of NAD+, KPV tripeptide, and sodium citrate.

19. An iontophoretic delivery system comprising the composition of claim 17.
